(19) United States
(12) Patent Application Publication
(10) Pub. No.: US 2018/0249917 A1
(43) Pub. Date: Sep. 6, 2018

(54) BIOLOGICAL INFORMATION ANALYSIS APPARATUS, BIOLOGICAL INFORMATION ANALYSIS SYSTEM, BEATING INFORMATION MEASUREMENT SYSTEM, AND BIOLOGICAL INFORMATION ANALYSIS PROGRAM

(71) Applicant: SEIKO EPSON CORPORATION, Tokyo (JP)

(72) Inventors: Hideo SASAHARA, Chino-shi (JP); Norio TERUUCHI, Shiojiri-shi (JP)

(73) Assignee: SEIKO EPSON CORPORATION, Tokyo (JP)

(21) Appl. No.: 15/540,186

(22) PCT Filed: Dec. 8, 2015
(86) PCT No.: PCT/JP2015/006111

§ 371 Date: Jun. 27, 2017

(30) Foreign Application Priority Data
Jan. 7, 2015 (JP) 2015-001357
Jan. 8, 2015 (JP) 2015-002417

(51) Int. Cl. A61B 5/024; A61B 5/11; A61B 5/00

(52) U.S. Cl. CPC A61B 5/02416; A61B 5/02438; A61B 5/1118; A61B 5/0833; A61B 5/7235; A61B 5/721; A61B 5/681

(57) ABSTRACT

A biological information analysis apparatus, a biological information analysis system, and a biological information analysis program, capable of easily calculating an estimated value of the maximum oxygen intake. A biological information analysis apparatus includes an information acquisition portion that acquires biological information of a user, body motion information indicating body motion of the user, and user information regarding the user, a storage unit that stores the acquired biological information, body motion information, and user information, a level determination portion that determines an exercise habit level of the user on the basis of the biological information and the body motion information in a predetermined period, and an estimation unit (estimated value calculation portion) that estimates the maximum oxygen intake of the user on the basis of the exercise habit level and the user information.

### TECHNICAL FIELD

The present invention relates to a biological information analysis apparatus, a biological information analysis system, a beating information measurement system, and a biological information analysis program.

### BACKGROUND ART

In the related art, a gas phase respiratory function examination system has been proposed which detects expiration data (for example, an oxygen concentration and a carbonic acid gas concentration) of a subject, and performs health management corresponding to numerical values based on the expiration data (refer to PTL 1, for example).

The gas phase respiratory function examination system disclosed in PTL 1 includes a treadmill, a mouthpiece (mask), a sampling tube, a flow sensor, an infrared CO₂ sensor, a zirconia type O₂ sensor, and a microcomputer. In a case where the maximum oxygen intake (VO₂max) is measured by using such a system, a subject runs on the treadmill in a state of mounting the mask on the mouth. The expired air of the subject is distributed to the flow sensor and the infrared CO₂ sensor via the sampling tube connected to the mask. The flow sensor measures a flow rate of the expired air, and the infrared CO₂ sensor detects a carbonic acid gas concentration. The expired air having passed through the infrared CO₂ sensor is guided to the zirconia type O₂ sensor, and an oxygen concentration is detected by the O₂ sensor so as to be transmitted to the microcomputer. The microcomputer calculates and measures the maximum oxygen intake on the basis of the flow rate, the carbonic acid gas concentration, and the oxygen concentration of the expired air, transmitted from the flow sensor, the infrared CO₂ sensor, and the zirconia type O₂ sensor.

### CITATION LIST

### Patent Literature

PTL 1: JP-A-2000-316834

### SUMMARY OF INVENTION

### Technical Problem

However, since the gas phase respiratory function examination system disclosed in PTL 1 includes the treadmill, the mask, and various sensors and is thus large-sized and expensive, the system is not easily introduced. Since a user has to perform exercise putting a burden on the body of their own, such as a gradual increase of a load, in a state of mounting the mask, in order to measure the maximum oxygen intake, there is a problem in that it is hard to measure the maximum oxygen intake of a feeble user or a user whose physical condition is not good.

The invention has been made in order to solve at least some of the above-described problems, and an object of their own is to provide a biological information analysis apparatus, a biological information analysis system, and a biological information analysis program, capable of easily calculating an estimated value of the maximum oxygen intake.

### Solution to Problem

A biological information analysis apparatus according to a first aspect of the invention includes an information acquisition unit that acquires biological information of a user, body motion information indicating body motion of the user, and user information regarding the user; a storage unit that stores the biological information, the body motion information, and the user information which have been acquired; a level determination unit that determines an exercise habit level of the user on the basis of the biological information and the body motion information in a predetermined period; and an estimation unit that estimates the maximum oxygen intake of the user on the basis of the exercise habit level and the user information.

Examples of the biological information may include pulse wave information (for example, a pulse rate and a pulse wave interval) derived from a pulse wave signal, and examples of the body motion information may include an acceleration value acting due to body motion of the user. Examples of the user information may include the age, the sex, and a body mass index (BMI) of the user, and the predetermined period may be a week as an example.

The exercise habit level may be, for example, a level indicated by numerical values which are set stepwise for an exercise habit of the user corresponding to the frequency of exercise or the intensity of performed exercise based on biological information and body motion information in the predetermined period.

As a method of estimating the maximum oxygen intake in the estimation unit, for example, a method using a so-called Jackson's formula (refer to Jackson A S, Blair S N, Mahar M T, Wier L T, Ross R M, Stuteville J E: Prediction of functional aerobic capacity exercise testing. Med Sci Sports Exerc 1990, 22: 863 to 870) may be used. In a case of employing a calculation method using the Jackson's formula, if the age, the sex, a height, and a weight of the user are acquired as the user information, and the exercise habit level (the numerical values “0” to “10”) is acquired, the maximum oxygen intake of the user can be estimated by assigning these values to the Jackson's formula. The estimated maximum oxygen intake will be referred to as the estimated value of the maximum oxygen intake in some cases.

According to the first aspect, it is possible to determine an exercise habit level of a user on the basis of biological information and body motion information of the user in a predetermined period, and also to estimate the maximum oxygen intake by employing a method of calculating an estimated value of the maximum oxygen intake using, for example, the above Jackson's formula on the basis of the determined exercise habit level and user information. Therefore, it is possible to easily estimate an estimated value of the maximum oxygen intake of a user without performing exercise applying a load to the user in a state of mounting a mask as in the gas phase respiratory function examination system disclosed in PTL 1. Since the maximum oxygen intake can be estimated even without using a treadmill, it is possible to estimate the maximum oxygen intake of the user with a relatively simple configuration.

In the gas phase respiratory function examination system disclosed in PTL 1, a user is required to perform exercise after wearing a mask in order to measure the maximum oxygen intake, and thus sanitary problems may occur. In contrast, in the first aspect, it is not necessary to wear a mask, and thus it is possible to estimate the maximum oxygen intake sanitarily.

In the first aspect, it is preferable that the biological information analysis apparatus further includes a presentation unit that presents a distribution of values of the maximum oxygen intake in a group which is classified on the basis of the user information.

The group may be, for example, not only a group having the same sex and age as those of a user, but also a group in which a period is the same after daily exercise is started, and a group of users using the same appliance or sensor (biological information analysis apparatus).

According to the first aspect, since a distribution of values of the maximum oxygen intake in the group is presented, it is possible to easily recognize the distribution in the group. If distributions of values of the maximum oxygen intake in a plurality of groups are presented, a user can compare the distributions of values of the maximum oxygen intake in the respective groups with each other.

In the first aspect, it is preferable that the presentation unit presents a position corresponding to an estimated value of the maximum oxygen intake of the user in the distribution.

According to the first aspect, the user can recognize a position of the estimated value of the maximum oxygen intake of the user in a distribution of values of the maximum oxygen intake in the group. Consequently, the user can objectively recognize the standing position of their own, that is, the estimated value of the maximum oxygen intake of their own in contrast to the others in the group. Therefore, it is possible to increase an exercise motivation of the user.

In the first aspect, it is preferable that the biological information analysis apparatus further includes an index calculation unit that calculates an exercise ability index indicating exercise ability of the user on the basis of the user information and the estimated value of the maximum oxygen intake; and an index presentation unit that presents the calculated exercise ability index.

The exercise ability index may include, for example, an expected time for long distance running (for example, a full marathon) of a user.

According to the first aspect, since an exercise ability index of a user can be presented, even a user not having the detailed knowledge about the maximum oxygen intake can easily recognize the exercise ability of their own. Consequently, it is possible to further increase an exercise motivation of the user.

In the first aspect, it is preferable that the biological information analysis apparatus further includes an index calculation unit that calculates an exercise ability index indicating exercise ability of the user on the basis of at least one of the biological information, the body motion information, and the exercise habit level; and an index presentation unit that presents the calculated exercise ability index.

According to the first aspect, since an exercise ability index of a user can be presented, even a user not having the detailed knowledge about the maximum oxygen intake can easily recognize the exercise ability of their own. Consequently, it is possible to further increase an exercise motivation of the user.

In the first aspect, it is preferable that the biological information analysis apparatus further includes a change information presentation unit that presents a change in the estimated value of the maximum oxygen intake of the user in correlation with the date and time on which the maximum oxygen intake is estimated.

According to the first aspect, since a change in an estimated value of the maximum oxygen intake of a user can be presented, the user can easily recognize an objective change in the maximum oxygen intake of their own. Therefore, it is possible to increase an exercise motivation of the user.

In the first aspect, it is preferable that the biological information analysis apparatus further includes a change information presentation unit that presents a change in the exercise ability index along with a change in the estimated value of the maximum oxygen intake of the user in correlation with the date and time on which the maximum oxygen intake is estimated.

According to the first aspect, since a change in an estimated value of the maximum oxygen intake and an exercise ability index can be presented together, even a user not having the detailed knowledge about the maximum oxygen intake can easily recognize a relationship between the exercise ability and the estimated value of the maximum oxygen intake of their own. Consequently, it is possible to further increase an exercise motivation of the user.

In the first aspect, it is preferable that the user information includes the age, the sex, a height, and a weight of the user.

In the first aspect, since the height and the weight of the user can be acquired, a BMI can be calculated. Consequently, even if a value of the BMI cannot be acquired as user information, it is possible to reliably calculate an estimated value of the maximum oxygen intake by using the above Jackson's formula. Therefore, it is possible to improve a user's convenience.

A biological information analysis system according to a second aspect of the invention includes the biological information analysis apparatus; and a detection apparatus that is mounted on the user, in which the detection apparatus includes a biological information detection unit that detects biological information of the user, a body motion information detection unit that detects body motion information of the user, and a detection apparatus side transmission unit that transmits the biological information and the body motion information to the biological information analysis apparatus.

According to the second aspect, it is possible to achieve the same effects as in the biological information analysis apparatus related to the first aspect. Regarding the biological information analysis apparatus, since biological information and body motion information of a user are detected by the detection apparatus, and are transmitted to the biological information analysis apparatus, the biological information analysis apparatus is not required to include a detection unit detecting the biological information and the body motion information. Therefore, since a user wears the detection apparatus, and can acquire biological information and body motion information for calculating an exercise habit level of the user, it is possible to more easily calculate an estimated value of the maximum oxygen intake than in a case where the biological information and the body motion information are acquired through an input operation performed by the user.

A biological information analysis program according to a third aspect of the invention is a biological information analysis program which is read to be executed by a computer, and causes the computer to function as the biological information analysis apparatus.

According to the third aspect, the program is executed by the computer, and thus the computer can achieve the same effects as in the biological information analysis apparatus related to the first aspect.

A beating information measurement system according to this application example includes a pulse wave detection unit that detects pulse wave information indicating a pulse wave; a heartbeat detection unit that detects heartbeat information indicating a heartbeat; an activity amount calculation unit that calculates an activity amount of the body; and a beating information determination unit that selects one of the pulse wave information and the heartbeat information on the basis of the activity amount, and determines beating information on the basis of the selected information, in which the beating information determination unit determines the beating information on the basis of the heartbeat information in a case where the activity amount exceeds a predetermined reference.

According to this configuration, beating information is determined on the basis of one of pulse wave information and heartbeat information, and the beating information is determined on the basis of the heartbeat information in a case where an activity amount of the body exceeds a predetermined reference. Therefore, in a case where an activity amount is large due to a vigorous motion, the beating information is determined on the basis of the heartbeat information regardless of the pulse wave information, and thus it is possible to prevent deterioration in reliability of beating information measurement.

In the beating information measurement system according to the application example, it is preferable that the beating information determination unit determines the beating information on the basis of the pulse wave information in a case where the activity amount does not exceed the predetermined reference.

According to this configuration, in a stable case in which an activity amount does not exceed the predetermined reference, the beating information is determined on the basis of the pulse wave information, and thus a mechanism for detecting the heartbeat information is not necessary, and it is possible to measure the beating information with a simple configuration.

In the beating information measurement system according to the application example, the beating information may be a heart rate or a pulse rate.

In the beating information measurement system according to the application example, it is preferable that the beating information determination unit includes a determination section that determines whether or not the heartbeat detection unit detects the heartbeat information.

According to this configuration, it is possible to determine whether or not the heartbeat detection unit detects the heartbeat information.

In the beating information measurement system according to the application example, it is preferable that the beating information determination unit determines the beating information on the basis of the heartbeat information in a case where the determination section determines that the heartbeat detection unit detects the heartbeat information, and the activity amount exceeds the predetermined reference.

According to this configuration, since the heartbeat detection unit detects heartbeat information, and determines beating information on the basis of the heartbeat information in a case where an activity amount exceeds a predetermined reference, it is possible to determine beating information based on the heartbeat information with high efficiency.

In the beating information measurement system according to the application example, it is preferable that the activity amount calculation unit calculates the activity amount on the basis of position information based on a location position and exercise information indicating exercise intensity based on body motion.

According to this configuration, it is possible to calculate an activity amount with high accuracy on the basis of position information and exercise information.

It is preferable that the beating information measurement system according to the application example further includes a display unit that displays the beating information, and the beating information determination unit displays information indicating whether the beating information is calculated on the basis of the heartbeat information or is calculated on the basis of the pulse wave information, on the display unit.

According to this configuration, it is possible to perform a notification of whether beating information is calculated on the basis of heartbeat information or the beating information is calculated on the basis of pulse wave information.

It is preferable that the beating information measurement system according to the application example further includes a calorie amount calculation unit that calculates a calorie consumption amount on the basis of the calculated beating information.

According to this configuration, it is possible to perform a notification of information regarding a calorie consumption amount in addition to beating information.

In the beating information measurement system, the pulse wave detection unit may detect the pulse wave at a limb part of a subject, and the heartbeat detection unit may detect the heartbeat at a trunk part of the subject.

The beating information measurement system may further include a pulse wave measurement apparatus and a heartbeat measurement apparatus which are communicably connected to each other, the pulse wave measurement apparatus may include the pulse wave detection unit, the activity amount calculation unit, and the beating information determination unit, and the heartbeat measurement apparatus may include the heartbeat detection unit.

The beating information measurement system may further include a pulse wave measurement apparatus, a heartbeat measurement apparatus, an information terminal apparatus which are communicably connected to each other, the pulse wave measurement apparatus may include the pulse wave detection unit and the activity amount calculation unit, the heartbeat measurement apparatus may include the heartbeat detection unit, and the information terminal apparatus may include the beating information determination unit.

A beating information measurement method includes a pulse wave detection step of detecting pulse wave information indicating a pulse wave; a heartbeat detection step of detecting heartbeat information indicating a heartbeat; an activity amount calculation step of calculating an activity amount of the body; a selection step of selecting one of the pulse wave information and the heartbeat information on the basis of the activity amount; and a beating information determination step of determining beating information on the basis of the selected information, in which, in the selection step, the beating information is determined on the basis of the heartbeat information in a case where the activity amount exceeds a predetermined reference.

According to this method, beating information is determined on the basis of one of pulse wave information and heartbeat information, and the beating information is determined on the basis of the heartbeat information in a case where an activity amount of the body exceeds a predetermined reference. Therefore, in a case where an activity amount is large due to a vigorous motion, the beating information is determined on the basis of the heartbeat information regardless of the pulse wave information, and thus it is possible to prevent deterioration in reliability of beating information measurement.

A beating information measurement apparatus includes a pulse wave detection unit that detects pulse wave information indicating a pulse wave; a communication unit that receives heartbeat information indicating a heartbeat; an activity amount calculation unit that calculates an activity amount of the body; and a beating information determination unit that selects one of the pulse wave information and the heartbeat information on the basis of the activity amount, and determines beating information on the basis of the selected information, in which the beating information determination unit determines the beating information on the basis of the heartbeat information in a case where the activity amount exceeds a predetermined reference.

According to this configuration, beating information is determined on the basis of one of pulse wave information and heartbeat information, and the beating information is determined on the basis of the heartbeat information in a case where an activity amount of the body exceeds a predetermined reference. Therefore, in a case where an activity amount is large due to a vigorous motion, the beating information is determined on the basis of the heartbeat information regardless of the pulse wave information, and thus it is possible to prevent deterioration in reliability of beating information measurement.

### BRIEF DESCRIPTION OF DRAWINGS

FIG. 1 is a schematic diagram illustrating a biological information analysis system according to a first embodiment of the invention.

FIG. 2 is a block diagram illustrating a configuration of a biological information detection apparatus in the first embodiment.

FIG. 3 is a block diagram illustrating a configuration of a control unit of the biological information detection apparatus in the first embodiment.

FIG. 4 is a block diagram illustrating a configuration of an information processing apparatus in the first embodiment.

FIG. 5 is a diagram illustrating zone setting information stored in a storage unit of the information processing apparatus in the first embodiment.

FIG. 6 is a diagram illustrating computation information stored in the storage unit of the information processing apparatus in the first embodiment.

FIG. 7 is a block diagram illustrating a configuration of a control unit of the information processing apparatus in the first embodiment.

FIG. 8 is a diagram illustrating an example of a user information registration screen in the first embodiment.

FIG. 9 is a diagram illustrating an example of an analysis result screen in the first embodiment.

FIG. 10 is a diagram illustrating an example of a change result screen in the first embodiment.

FIG. 11 is a diagram illustrating an example of a message display screen displayed on a display unit of the biological information detection apparatus in the first embodiment.

FIG. 12 is a schematic diagram illustrating a biological information analysis system according to a second embodiment of the invention.

FIG. 13 is a diagram illustrating an example of an analysis result screen in the second embodiment.

FIG. 14 is a block diagram illustrating a schematic configuration of a beating information measurement system according to a third embodiment of the invention.

FIG. 15 is a diagram illustrating a hardware configuration of a pulse wave measurement apparatus.

FIG. 16 is a diagram illustrating a detailed functional configuration of a measurement control unit.

FIG. 17 is a flowchart illustrating a flow of a beating information measurement process.

FIG. 18 is a block diagram illustrating a schematic configuration of a beating information measurement system according to a fourth embodiment of the invention.

### DESCRIPTION OF EMBODIMENTS

### First Embodiment

Hereinafter, a first embodiment of the invention will be described with reference to the drawings.

[Schematic Configuration of Biological Information Analysis System]

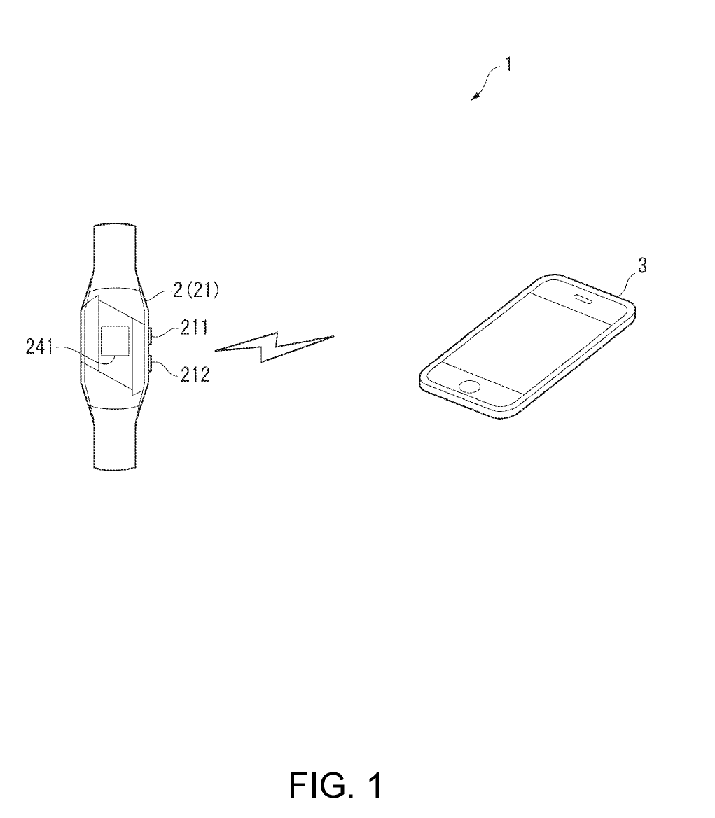

FIG. 1 is a schematic diagram illustrating a biological information analysis system 1 according to the present embodiment.

As illustrated in FIG. 1, the biological information analysis system 1 according to the present embodiment includes a biological information detection apparatus (hereinafter, referred to as a detection apparatus in some cases) 2 and an information processing apparatus 3.

In the biological information analysis system 1, the detection apparatus 2 detects biological information and body motion information and transmits the information to the information processing apparatus 3. The information processing apparatus 3 calculates an estimated value of the maximum oxygen intake and an exercise ability index on the basis of not only the received biological information and body motion information but also user information which is input by a user, and presents the calculation results to the user. If presentation information (for example, message information based on the maximum oxygen intake of the user which will be described later) is received from the information processing apparatus 3, the detection apparatus 2 notifies the user of the presentation information.

Hereinafter, each configuration of the biological information analysis system 1 will be described.

### [Configuration of Detection Apparatus]

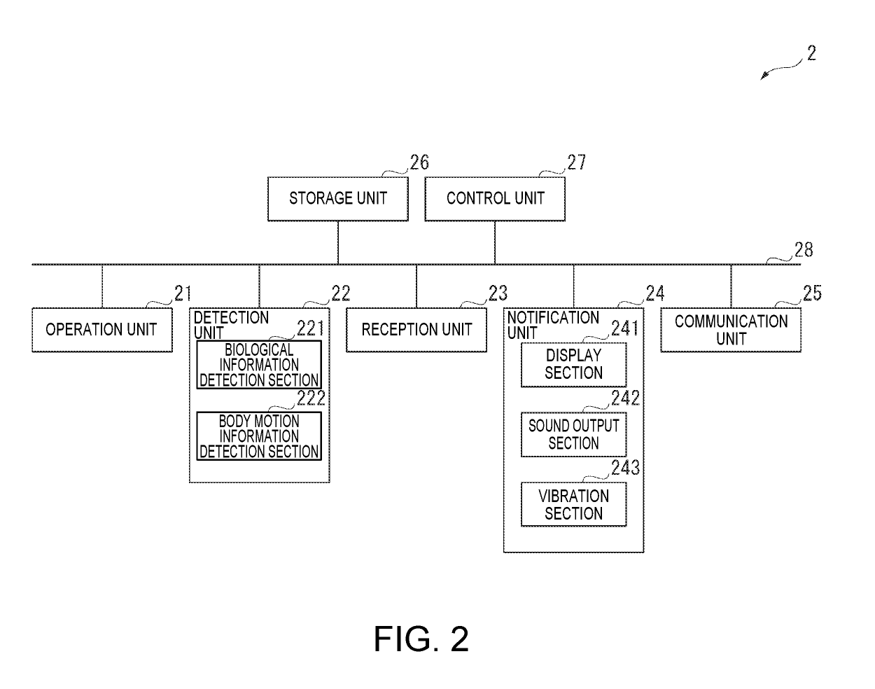

FIG. 2 is a block diagram illustrating a configuration of the biological information detection apparatus 2.

The biological information detection apparatus 2 corresponds to a detection apparatus of the invention, and is a wearable apparatus which is mounted on the wrist of the user, and detects and stores the biological information and the body motion information. The detection apparatus 2 not only transmits the detected and stored biological information and body motion information to the information processing apparatus 3 but also notifies the user of the presentation information received from the information processing apparatus 3, as described above.

As illustrated in FIG. 2, the detection apparatus 2 includes an operation unit 21, a detection unit 22, a reception unit 23, a notification unit 24, a communication unit 25, a storage unit 26, and a control unit 27, and the respective units 21 to 27 are electrically connected to each other via a bus line 28.

### [Configuration of Operation Unit]

The operation unit 21 receives an input operation performed by the user, and outputs an operation signal corresponding to the input operation to the control unit 27. The operation unit 21 outputs, to the control unit 27, an operation signal corresponding to an input operation on buttons 211 and 212 (refer to FIG. 1) provided to be exposed to an outer surface of a casing of the detection apparatus 2. The operation unit 21 may be configured to recognize a voice of the user (a sound of the user) and to output an operation signal corresponding to the voice to the control unit 27. The operation unit 21 may be configured to detect a tapping operation of the user and to output an operation signal corresponding to the tapping operation to the control unit 27.

### [Configuration of Detection Unit]

The detection unit 22 includes a biological information detection section 221 and a body motion information detection section 222 which respectively detect biological information and body motion information of the user.

The biological information detection section 221 detects biological information of the user wearing the detection apparatus 2. In the present embodiment, the biological information detection section 221 includes a pulse wave sensor which detects a pulse wave as biological information and outputs a pulse wave signal indicating the pulse wave. The biological information detection section 221 may be configured to detect a heartbeat instead of a pulse wave as biological information, and may be configured to detect blood pressure, body temperature, a blood sugar level, and the like, in addition thereto.

The body motion information detection section 222 detects an acceleration which changes according to body motion of the user as body motion information of the user. In the present embodiment, the body motion information detection section 222 includes an acceleration sensor which detects an acceleration value (acceleration data) acting according to body motion of the user as body motion information.

### [Configuration of Reception Unit]

The reception unit 23 acquires position information indicating the present position of the detection apparatus 2 (that is, position information indicating the present position of the user). For example, the reception unit 23 is compatible with a satellite positioning system such as a global positioning system (GPS), and acquires the position information indicating the present position on the basis of a radio wave transmitted from a satellite. The reception unit 23 outputs the acquired position information to the control unit 27. The position information is used for an analysis portion 366 which will be described later to calculate a running distance, a running speed, and the like of the user.

### [Configuration of Notification Unit]

The notification unit 24 performs a notification of various pieces of information under the control of the control unit 27. For example, the notification unit 24 performs a notification of an operation state of the detection apparatus 2, or detected information and message information received from the information processing apparatus 3. The notification unit 24 includes a display section 241, a sound output section 242, and a vibration section 243.

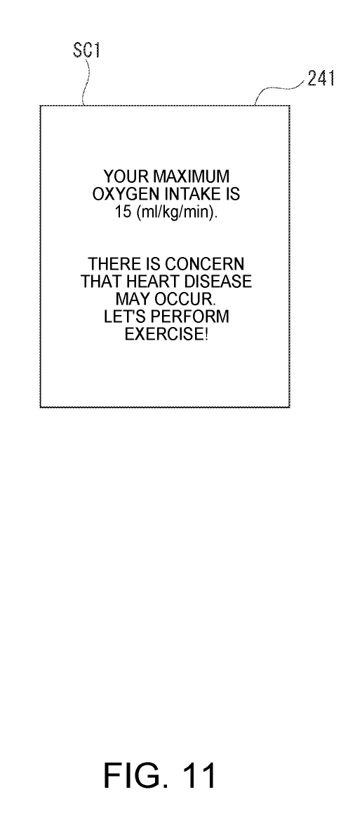

The display section 241 has an information display function by using liquid crystal or the like, and displays the various pieces of information. The display section 241 displays, for example, a menu screen or a message display screen (refer to FIG. 11) under the control of the control unit 27 which will be described later.

The sound output section 242 is configured to include a speaker, and outputs a sound corresponding to sound information which is input from the control unit 27. For example, the sound output section 242 outputs a message display sound when the display section 241 displays a message display screen.

The vibration section 243 includes a motor whose driving is controlled by the control unit 27 which will be described later, and performs a notification of a state of the biological information detection apparatus 2 through vibration caused by driving of the motor.

### [Configuration of Communication Unit]

The communication unit 25 corresponds to a detection apparatus side transmission unit of the invention, and includes a communication module which can perform communication with the information processing apparatus 3 or the like. The communication unit 25 transmits detected and acquired biological information, body motion information, and position information to the information processing apparatus 3. In the present embodiment, the communication unit 25 performs communication with the information processing apparatus 3 in a wireless manner according to a short-range radio communication method, but may perform communication with the information processing apparatus 3 via a relay device such as a cradle, or may be perform communication with the information processing apparatus 3 via a cable. The communication unit 25 may perform communication with an external apparatus such as the information processing apparatus 3 via a network.

### [Configuration of Storage Unit]

The storage unit 26 is formed of a storage device including a flash memory or the like, and stores programs and data required for an operation of the detection apparatus 2.

For example, the storage unit 26 stores in advance connection information for performing communication connection to the information processing apparatus 3 via the communication unit 25 as the data.

The storage unit 26 stores biological information and body motion information detected by the detection unit 22 and position information acquired by the reception unit 23 under the control of the control unit 27. The storage unit 26 stores presentation information (message information) which is acquired from the information processing apparatus 3 via the communication unit 25.

### [Configuration of Control Unit]

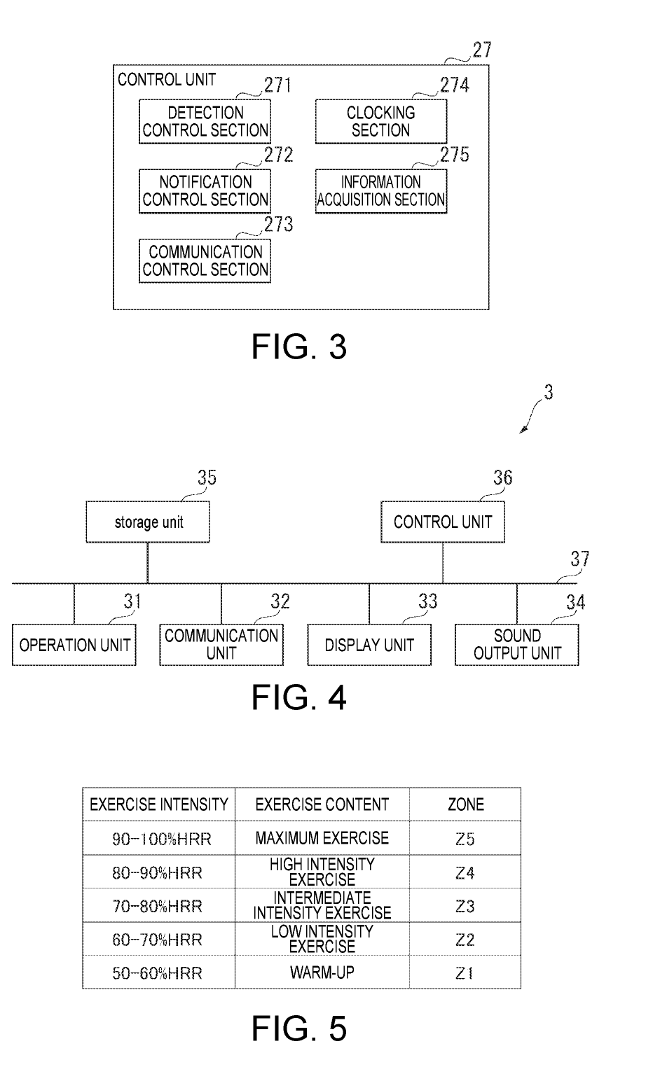

FIG. 3 is a block diagram illustrating a configuration of the control unit 27.

The control unit 27 is configured to include a processing circuit such as a central processing unit (CPU), and controls an operation of the detection apparatus 2 autonomously or in response to an operation signal which is input from the operation unit 21 according to an operation of the user. The control unit 27 acquires, for example, biological information and body motion information detected by the detection unit 22 or position information acquired by the reception unit 23, and transmits the information to the information processing apparatus 3.

The control unit 27 includes a detection control section 271, a notification control section 272, a communication control section 273, a clocking section 274, and an information acquisition section 275 as functional sections which are realized by the processing circuit executing the program stored in the storage unit 26 as illustrated in FIG. 3.

The detection control section 271 controls an operation of the detection unit 22, and stores a detection result in the detection unit 22 in the storage unit 26. The detection control section 271 causes the detection unit 22 to detect each piece of information if the detection unit 22 is in a state of being capable of detecting biological information and body motion information. On the other hand, the detection control section 271 stops an operation of the detection unit 22 and can thus reduce power consumption if the detection unit 22 is in a state of not being capable of detecting each piece of information.

The notification control section 272 controls an operation of the notification unit 24. For example, the notification control section 272 controls an operation of the display section 241 of the notification unit 24, and displays biological information and body motion information detected by the detection unit 22.

If message information is received from the information processing apparatus 3, the notification control section 272 causes the notification unit 24 to perform a notification of the message information. For example, the notification control section 272 displays a message display screen including a message corresponding to the message information on the display section 241. For example, the notification control section 272 causes the sound output section 242 to output a message display sound and causes the vibration section 243 to vibrate. Consequently, the user can recognize that there is a high probability that heart disease may occur in the user.

The communication control section 273 controls the communication unit 25 to perform communication with the information processing apparatus 3 on the basis of the connection information stored in the storage unit 26.

The clocking section 274 measures the present date and time.

The information acquisition section 275 acquires biological information and body motion information detected by the detection unit 22, and position information acquired by the reception unit 23, and stores the information in the storage unit 26. At this time, the information acquisition section 275 stores the detection date and time and the acquisition date and time of the information by referring to the present date and time measured by the clocking section 274 along with the biological information, the body motion information, and the position information.

The biological information, the body motion information, the position information acquired and stored in the above-described way are transmitted to the information processing apparatus 3 by the communication unit 25 under the control of the communication control section 273.

### [Configuration of Information Processing Apparatus]

FIG. 4 is a block diagram illustrating a configuration of the information processing apparatus 3.

The information processing apparatus 3 corresponds to a biological information analysis apparatus of the invention, and is formed of, for example, a smart phone (multifunction mobile phone), a tablet PC, a personal computer (PC), or the like. As mentioned above, the information processing apparatus 3 calculates (determines) exercise intensity of the user based on the biological information and the body motion information received from the detection apparatus 2, and an exercise habit level of the user based on the exercise intensity and exercise time. The information processing apparatus 3 estimates the maximum oxygen intake on the basis of user information which is input to a displayed registration screen and the exercise habit level. The information processing apparatus 3 transmits message information including a message corresponding to the estimated maximum oxygen intake, and also displays the message information. The information processing apparatus 3 displays an exercise ability index based on the maximum oxygen intake in response to the user's operation.

As illustrated in FIG. 4, the information processing apparatus 3 includes an operation unit 31, a communication unit 32, a display unit 33, a sound output unit 34, a storage unit 35, and a control unit 36, and the respective units are connected to each other via a bus line 37.

### [Configuration of Operation Unit]

The operation unit 31 receives an input operation performed by the user, and outputs operation information corresponding to the input operation to the control unit 36. The operation unit 31 is formed of, for example, a physical key or a touch panel provided in a casing of the information processing apparatus 3, and may be formed of a keyboard, a pointing device, and the like connected to the information processing apparatus 3 in a wired or wireless manner.

### [Configuration of Communication Unit]

The communication unit 32 includes a first communication module which can perform communication with an external apparatus such as the detection apparatus 2 and a second communication module which can perform communication with a server (not illustrated) on a network such as the Internet, and performs communication with the external apparatus and the server under the control of the control unit 36. In a case where the communication unit 32 can perform communication with each of the external apparatus and the server in the same communication method, the communication unit 32 may include one of the first communication module and the second communication module, and the second communication module may not be provided if communication with the server is not necessary.

### [Configuration of Display Unit and Sound Output Unit]

The display unit 33 corresponds to a range presentation unit, an index presentation unit, and a change information presentation unit. The display unit 33 may be formed of, for example, various display panels such as a liquid crystal display panel, an organic electroluminescence (EL) panel, and an electrophoretic panel, and displays an image generated by a presentation information generation portion 370 which will be described later. Specifically, the display unit 33 displays execution screens (for example, an execution screen ES which will be described later) of an operating system (OS) and various applications executed by the control unit 36.

The sound output unit 34 is configured to include a speaker, and outputs a sound corresponding to sound information which is input from the control unit 36. For example, in a case where the control unit 36 executes an information management application which will be described later, the sound output unit 34 outputs a sound corresponding to information presented to the user.

### [Configuration of Storage Unit]

The storage unit 35 is formed of storage devices such as a solid state drive (SSD), a hard disk drive (HDD), and a flash memory, and stores programs and data required for an operation of the information processing apparatus 3. As the programs, the storage unit 35 stores the operating system (OS) controlling the information processing apparatus 3 and also stores a biological information analysis application (hereinafter, referred to as an analysis application in some cases) which will be described later.

The storage unit 35 stores not only various pieces of information received from the detection apparatus 2, the input content on a registration screen which will be described later, but also zone setting information and computation information used by the analysis portion 366 and a level determination portion 367 which will be described later.

### [Zone Setting Information]

FIG. 5 is a diagram illustrating an example of zone setting information stored in the storage unit 35.

The storage unit 35 stores the following zone setting information. The zone setting information is a table in which exercise intensity of exercise performed by the user is distributed to a plurality of divisions called zones.

Specifically, in the zone setting information, as illustrated in FIG. 5, the zone is divided into a zone (Z1) in which % HRR (heart rate reserved) indicating exercise intensity is 50 or more and below 60, a zone (Z2) in which % HRR is 60 or more and below 70, a zone (Z3) in which % HRR is 70 or more and below 80, a zone (Z4) in which % HRR is 80 or more and below 90, and a zone (Z5) in which % HRR is 90 or more and 100 or less, and the exercise content and the zone names are set.

Specifically, in the zone in which % HRR is 50 or more and below 60, “warm-up” is set as the exercise content, and “Z1” is set as the zone name. In the zone in which % HRR is 60 or more and below 70, “low intensity exercise” is set as the exercise content, and “Z2” is set as the zone name. In the zone in which % HRR is 70 or more and below 80, “intermediate intensity exercise” is set as the exercise content, and “Z3” is set as the zone name. In the zone in which % HRR is 80 or more and below 90, “high-intensity exercise” is set as the exercise content, and “Z4” is set as the zone name. In the zone in which % HRR is 90 or more and 100 or less, “maximum intensity exercise” is set as the exercise content, and “Z5” is set as the zone name.

In the present embodiment, regarding the exercise intensity, the zone is set on the basis of % HRR indicating exercise intensity based on the Karvonen method, but, this is only an example, and, the zone may be set on the basis of % MHR (maximum heart rate).

### [Computation Information]

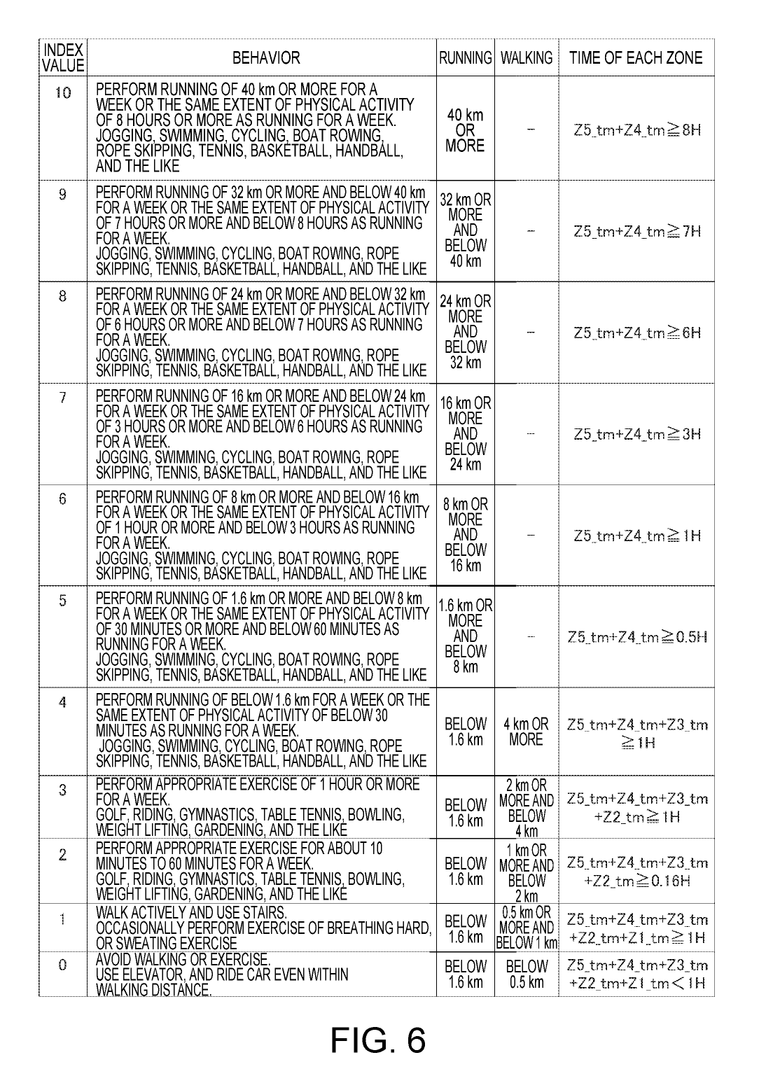

FIG. 6 is a diagram illustrating an example of computation information stored in the storage unit 35.

The storage unit 35 stores the following computation information.

The computation information is information for calculating an exercise habit level expressed as an index value when the maximum oxygen intake of the user is estimated by the control unit 36 which will be described later.

Here, the exercise habit level will be described. The exercise habit level is a level indicated by numerical values which are set stepwise for an exercise habit calculated on the basis of the frequency of exercise of the user or the intensity of performed exercise based on biological information and body motion information in a predetermined period (for example, for one week). In the present embodiment, the exercise habit level is set in eleven stages, and numerical values indicating the level are set to “0” to “10”.

A classification of the level based on the exercise (behavior) content, a running distance, and a walking distance corresponding to each exercise habit level described below is set in advance on the basis of a physical activity level (PAL) and a physical activity rating (PAR).

In the computation information for calculating such an exercise habit level, for example, as illustrated in FIG. 6, for each of exercise habit levels of “0 to 10”, conditions for the behavior content, a running distance, and a walking distance corresponding to an exercise habit level, and a condition for a total value of exercise time in the zone for a week are set.

Specifically, as a behavior of the user corresponding to the exercise habit level “10”, “running of 40 km or more for a week or the same extent of physical activity of 8 hours or more as the running for a week” is set. Specific examples of the physical activity may include “jogging, swimming, cycling, boat rowing, rope skipping, tennis, basketball, and handball”.

“40 km or more” is set in a running distance in the exercise habit level. As a condition for the total time, the content that “a total value of exercise time of the zone Z5 and exercise time of the zone Z4 is 8 hours or more” is set.

As a behavior of the user corresponding to the exercise habit level “9”, “running of 32 km or more and below 40 km for a week or the same extent of physical activity of 7 hours or more and below 8 hours as the running for a week” is set. “32 km or more and below 40 km” is set in a running distance in the exercise habit level. As a condition for the total time, the content that “a total value of exercise time of the zone Z5 and exercise time of the zone Z4 is 7 hours or more” is set.

As a behavior of the user corresponding to the exercise habit level “8”, “running of 24 km or more and below 32 km for a week or the same extent of physical activity of 6 hours or more and below 7 hours as the running for a week” is set. “24 km or more and below 32 km” is set in a running distance in the exercise habit level. As a condition for the total time, the content that “a total value of exercise time of the zone Z5 and exercise time of the zone Z4 is 6 hours or more” is set.

As a behavior of the user corresponding to the exercise habit level “7”, “running of 16 km or more and below 24 km for a week or the same extent of physical activity of 3 hours or more and below 6 hours as the running for a week” is set. “16 km or more and below 24 km” is set in a running distance in the exercise habit level. As a condition for the total time, the content that “a total value of exercise time of the zone Z5 and exercise time of the zone Z4 is 3 hours or more” is set.

As a behavior of the user corresponding to the exercise habit level “6”, “running of 8 km or more and below 16 km for a week or the same extent of physical activity of 1 hour or more and below 3 hours as the running for a week” is set. “18 km or more and below 16 km” is set in a running distance in the exercise habit level. As a condition for the total time, the content that “a total value of exercise time of the zone Z5 and exercise time of the zone Z4 is 1 hour or more” is set.

As a behavior of the user corresponding to the exercise habit level “5”, “running of 1.6 km or more and below 8 km for a week or the same extent of physical activity of 30 minutes or more and below 60 minutes as the running for a week” is set. “1.6 km or more and below 8 km” is set in a running distance in the exercise habit level. As a condition for the total time, the content that “a total value of exercise time of the zone Z5 and exercise time of the zone Z4 is 0.5 hours or more” is set.

As a behavior of the user corresponding to the exercise habit level “4”, “running of below 1.6 km for a week or the same extent of physical activity of below 30 minutes as the running for a week” is set. “Below 1.6 km” is set in a running distance of the user, and “4 km or more” is set in a walking distance. As a condition for the total time, the content that “a total value of exercise time of the zone Z5, exercise time of the zone Z4, and exercise time of the zone Z3 is 1 hour or more” is set.

As a behavior of the user corresponding to the exercise habit level “3”, “appropriate exercise of 1 hour or more for a week” is set. Examples of the appropriate exercise may include golf, riding, gymnastics, table tennis, bowling, weight lifting, and gardening.

“Below 1.6 km” is set in a running distance in the exercise habit level, and “2 km or more and below 4 km” is set in a walking distance. As a condition for the total time, the content that “a total value of exercise time of the zone Z5, exercise time of the zone Z4, exercise time of the zone Z3, and exercise time of the zone Z2 is 1 hour or more” is set.

As a behavior of the user corresponding to the exercise habit level “2”, “appropriate exercise of 10 minutes or more and below 60 minutes for a week” is set. “Below 1.6 km” is set in a running distance in the exercise habit level, and “1 km or more and below 2 km” is set in a walking distance. As a condition for the total time, the content that “a total value of exercise time of the zone Z5, exercise time of the zone Z4, exercise time of the zone Z3, and exercise time of the zone Z2 is 0.16 hours or more” is set.

As a behavior of the user corresponding to the exercise habit level “1”, “a behavior of actively using stairs, a behavior of occasionally breathing hard, or sweating exercise” is set. “Below 1.6 km” is set in a running distance in the exercise habit level, and “0.5 km or more and below 1 km” is set in a walking distance. As a condition for the total time, the content that “a total value of exercise time of all of the zones Z1 to Z5 is 1 hour or more” is set.

As a behavior of the user corresponding to the exercise habit level “0”, “a behavior of avoiding walking or exercise, a behavior of using an elevator, or a behavior of riding a car even within walking distance” is set. “Below 1.6 km” is set in a running distance in the exercise habit level, and “below 0.5 km” is set in a walking distance. As a condition for the total time, the content that “a total value of exercise time of all of the zones Z1 to Z5 is below 1 hour” is set.

An exercise habit level is determined by the control unit 36 (specifically, the level determination portion 367) which will be described later on the basis of the computation information and at least one of a behavior, a running distance, and a walking distance of the user for a week, and a total value of exercise time of the zone.

The behavior, the running distance, and the walking distance allocated to each exercise habit level, and the total value of exercise time of each zone are only examples, and may be changed as appropriate in accordance with user information, a state of the user, and an estimation formula for estimating the maximum oxygen intake.

### [Configuration of Control Unit]

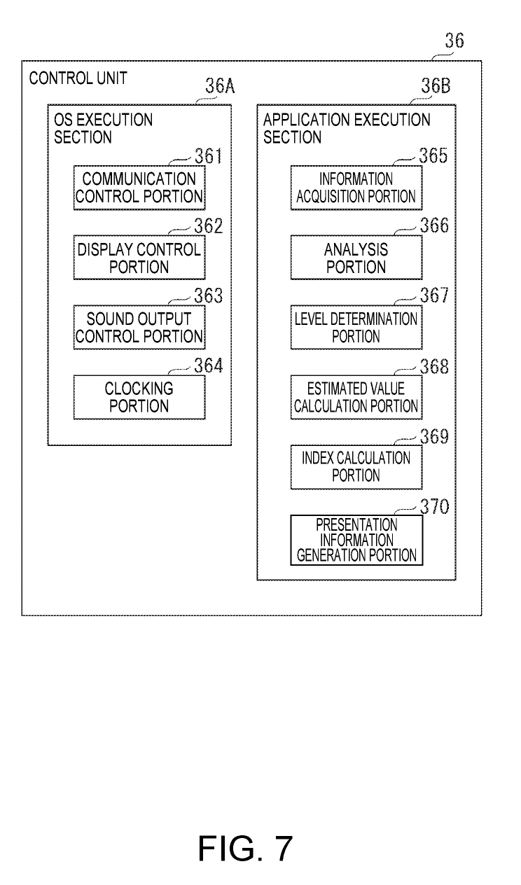

FIG. 7 is a block diagram illustrating a configuration of the control unit 36.

The control unit 36 is configured to include a central processing unit (CPU), and controls an operation of the information processing apparatus 3 by executing the program stored in the storage unit 35. The control unit 36 includes an OS execution section 36A and an application execution section 36B.

The OS execution section 36A is a functional section executing the OS stored in the storage unit 35, and includes a communication control portion 361, a display control portion 362, a sound output control portion 363, and a clocking portion 364.

The communication control portion 361 controls the communication unit 32 so as to perform communication with the external apparatus or the server.

The display control portion 362 displays the execution screen or execution screens (execution screens generated by other constituent elements) of other applications or the OS on the display unit 33.

The sound output control portion 363 outputs sound information regarding a sound which is output when the OS or the application is executed, to the sound output unit 34.

The clocking portion 364 measures the present date and time.

The application execution section 36B executes an application indicated by the OS execution section 36A according to operation information which is input from the operation unit 31, among the applications stored in the storage unit 35.

The application execution section 36B executes the analysis application stored in the storage unit 35 so as to function as and include an information acquisition portion 365, the analysis portion 366, the level determination portion 367, an estimated value calculation portion 368, an index calculation portion 369, and the presentation information generation portion 370.

### [Configuration of Information Acquisition Portion]

The information acquisition portion 365 acquires information which is input on an analysis application execution screen (user information registration screen) which will be described later by the user. The information acquisition portion 365 acquires various pieces of information (biological information, body motion information, position information, and date-and-time information) from the detection apparatus 2 via the communication unit 32, and stores and registers the information in the storage unit 35.

### [User Information Registration Screen]

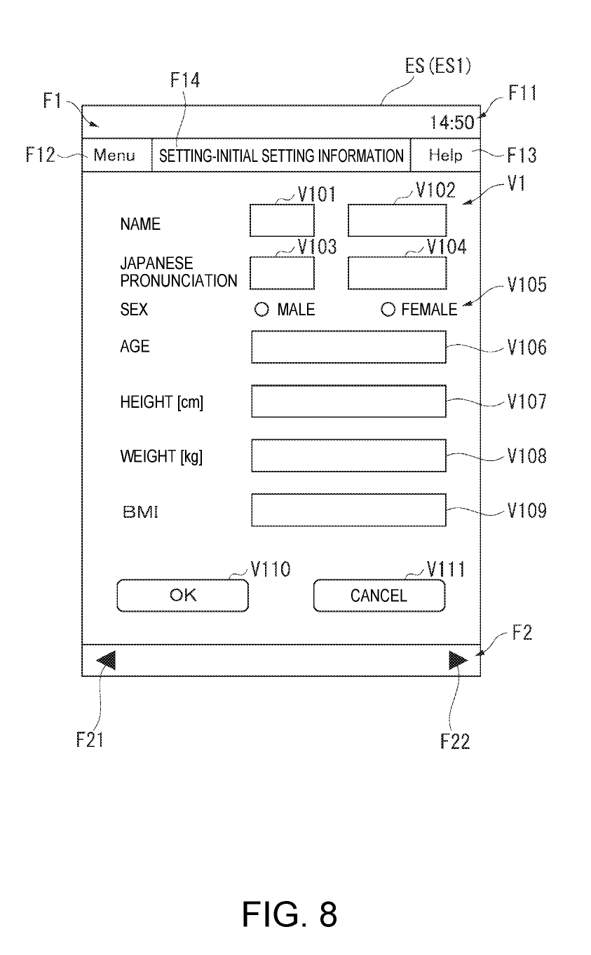

FIG. 8 is a diagram illustrating an example of a user information registration screen ES1.

The user information registration screen ES1 is one of the execution screens ES displayed when the analysis application is executed, and is a screen for inputting and registering the user information. As illustrated in FIG. 8, in the user information registration screen ES1, fixed display regions F1 and F2 are respectively set on a screen upper part and a screen lower part, and a variable display region V1 is set therebetween.

A time display region F11 in which the present time measured by the clocking portion 364 is set is disposed at an upper end of the fixed display region F1 on the screen upper part. A button F12 which transitions to a menu screen (not illustrated) if pressed (input) is disposed on the left at the lower end of the fixed display region F1, and a button F13 which transitions to a help screen (not illustrated) if pressed is disposed on the right. A title F14 indicating the content of the screen is disposed in a region interposed between the buttons F12 and F13.

Buttons F21 and F22 are disposed on the left and right of the fixed display region F2 on the screen lower part. The buttons F21 and F22 are buttons for causing a screen to transition.

Each input column for user information (the user's name) and the initial setting information is provided in the variable display region V1.

Specifically, the variable display region V1 is provided with surname input columns V101 and V103 for inputting the surname of the user in Chinese letters and hiragana letters, and first name input columns V102 and V104 for inputting the first name of the user in Chinese letters and hiragana letters, among the pieces of user information.

Input columns V105 to V109 for inputting the sex, the age, a weight, and a BMI of the user among the pieces of initial setting information, a registration button V110, and a cancel button V111 are disposed in the variable display region V1. Among the columns, radio buttons for selecting “male” and “female” are provided in the sex input column V105 for inputting the sex of the user.

If the registration button V110 is pressed, the content which is input to each of the input columns V101 to V109 is acquired by the information acquisition portion 365 so as to be stored in the storage unit 35.

The BMI can be calculated on the basis of a height and a weight as described above. Thus, if a height and a weight are respectively input to the height input column V107 and the weight input column V108, a BMI based on the input height and weight is set in the BMI input column V109.

When the input content is registered, a body type included in the initial setting information is determined on the basis of the set BMI and is registered.

On the other hand, if the cancel button V111 is pressed, return to a screen (for example, a menu screen) which is displayed right before the user information registration screen ES1 occurs.

The user information which is input to the user information registration screen ES1 is acquired by the information acquisition portion 365 as described above.

### [Configuration of Analysis Portion]

Referring to FIG. 7 again, the analysis portion 366 analyzes the biological information, the body motion information, the position information, and the date-and-time information received from the detection apparatus 2, and generates analysis results such as calorie consumption, a running distance, a walking distance, the number of steps, the exercise intensity (zones Z1 to Z5), and exercise time.

In the present embodiment, the analysis process in the analysis portion 366 is performed every week after the user starts to use the detection apparatus 2, but the period may be changed as appropriate.

The level determination portion 367 determines an exercise habit level of the user on the basis of biological information, body motion information, and position information, and the computation information in a predetermined period (for example, for a week).

Specifically, the level determination portion 367 detects in which zone of the zones Z1 to Z5 exercise is included by using biological information, body motion information, exercise intensity, and exercise time of the user on the basis of the zone setting information and the computation information stored in the storage unit 35, and determines an exercise habit level (any one of “0” to “10”) by using a total value of exercise times of the respective zones Z1 to Z5.

For example, in a case where the user has performed, within a week, the exercise of the zone Z5 for 1 hour, the exercise of the zone Z4 for 5 hours, the exercise of the zone Z3 for 2 hours, the exercise of the zone Z2 for 1 hour, and the exercise of the zone Z1 for 0.5 hours, an exercise habit level of the user is determined as being “8” on the basis of the exercise time of each zone in the computation information.

In a case where the analysis portion 366 calculates a running distance and a walking distance of the user on the basis of a movement distance and a movement speed based on a change in position information in the predetermined period, the level determination portion 367 may determine an exercise habit level on the basis of the running distance and the walking distance.

The display control portion may display an exercise content input screen for the user inputting the exercise content performed in a predetermined period on the display unit, and the level determination portion 367 may determine an exercise habit level of the user on the basis of the exercise content which is input by the user and the behavior content corresponding to each exercise habit level included in the computation information.

The estimated value calculation portion 368 corresponds to an estimation unit of the invention, and estimates the maximum oxygen intake of the user on the basis of the exercise habit level determined by the level determination portion 367 and the user information which is input by the user. The user information is the age, the sex, and the BMI of the user, and is stored in the storage unit 35 through an input operation of the user. The estimated value calculation portion 368 calculates an estimated value of the maximum oxygen intake by assigning the exercise habit level (for example, “8”), and the age, the sex, and the BMI of the user to the above Jackson's formula.

The age, the sex, and the BMI of the user are input on a user information registration screen which is generated by the presentation information generation portion 370 and is displayed on the display unit 33, through an operation performed by the user.

The index calculation portion 369 calculates an exercise ability index indicating the exercise ability of the user on the basis of the biological information, the body motion information, the exercise habit level, the user information, and the estimated value of the maximum oxygen intake.

In the present embodiment, the index calculation portion 369 calculates a full marathon expected time of the user as the exercise ability index. For example, the index calculation portion 369 calculates the full marathon expected time of the user on the basis of a running speed and an estimated value of the maximum oxygen intake which are calculated by using a height or a weight of the user, and a running distance and exercise time (running time) of the user.

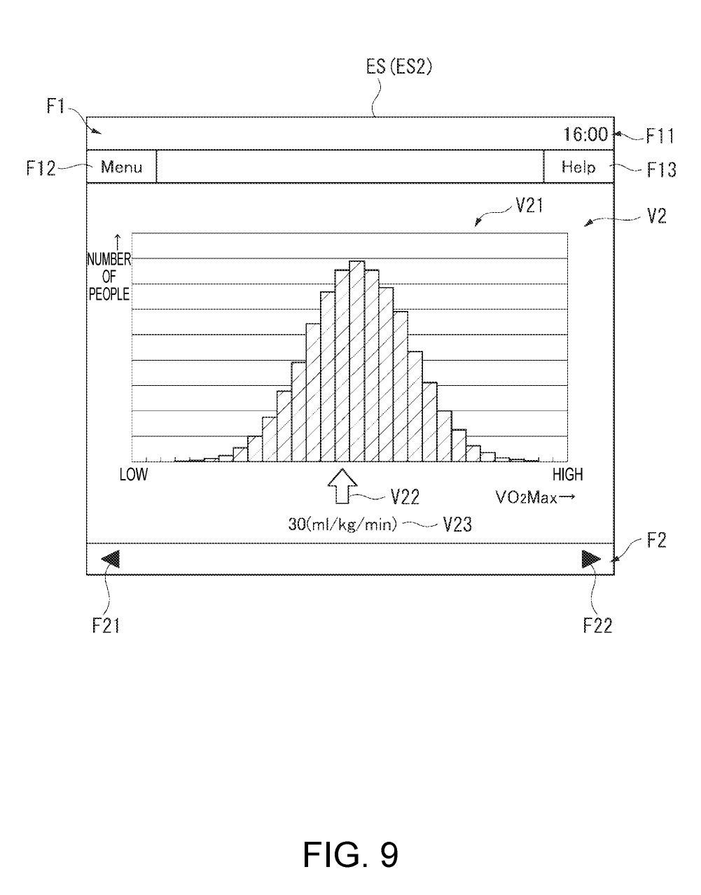

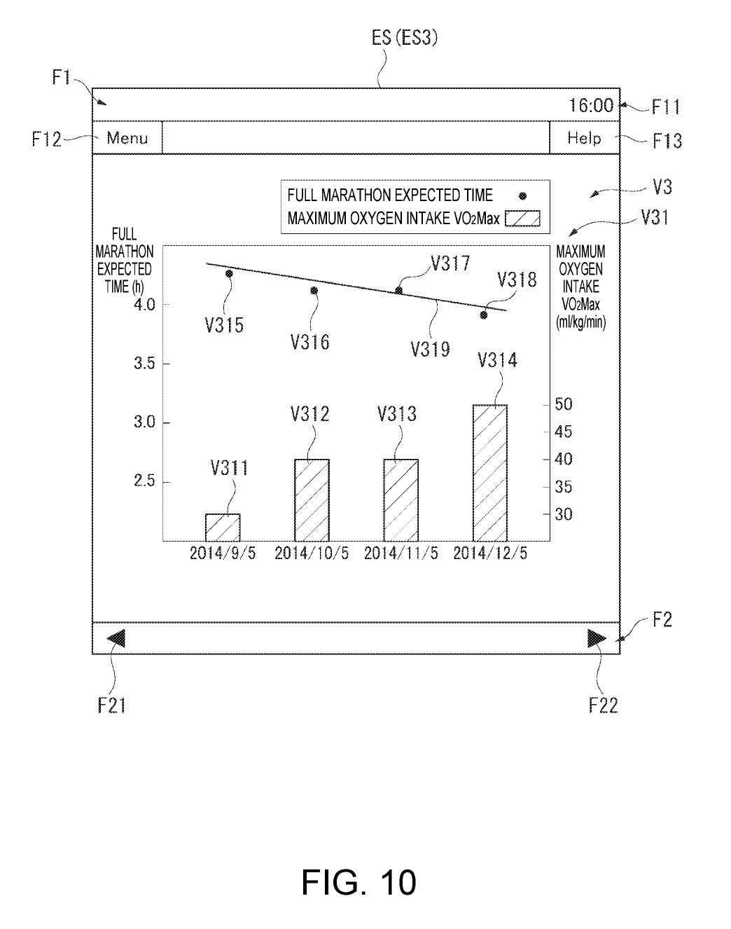

The presentation information generation portion 370 generates the execution screen ES of the analysis application. For example, the presentation information generation portion 370 generates not only the user information registration screen ES1 (refer to FIG. 8) but also an analysis result screen ES2 (refer to FIG. 9) obtained by analyzing an estimated value of the maximum oxygen intake of the user. The presentation information generation portion 370 also generates a change result screen ES3 (refer to FIG. 10) including a change in the estimated value of the maximum oxygen intake and a change in a full marathon expected time. These various screens are displayed on the display unit 33 under the control of the display control portion 362.

### [Analysis Result Screen]

FIG. 9 is a diagram illustrating an example of the analysis result screen ES2.

The analysis result screen ES2 is a screen included in the execution screens ES, and is a screen indicating a position corresponding to an estimated value of the maximum oxygen intake of the user in a distribution of values of the maximum oxygen intake of people corresponding to predetermined conditions which are set on the basis of the user information. Specifically, the analysis result screen ES2 indicates a position of an estimated value of the maximum oxygen intake of the user in a distribution of values of the maximum oxygen intake of people having the same age and sex as those of the user, that is, a position corresponding to an estimated value of the maximum oxygen intake of the user. As illustrated in FIG. 9, in the analysis result screen ES2, fixed display regions F1 and F2 are respectively set on a screen upper part and a screen lower part, and a variable display region V2 is set therebetween.

Information regarding a distribution of values of the maximum oxygen intake in the age and the sex of the user may be acquired by the information acquisition portion 365 from a server (not illustrated) or the like via the communication unit 32, and may be stored in the storage unit 35 in advance.

A graph V21 representing a distribution of values of the maximum oxygen intake of a group having the same age and sex as those of the user is displayed in the variable display region V2. As illustrated in FIG. 9, the number of people of the group is set on a longitudinal axis of the graph V21, and this indicates that the number of people increases toward the upper part of the graph V21. A value of the maximum oxygen intake is set on a transverse axis of the graph V21, and this indicates that the value of the maximum oxygen intake increases toward the right of the graph V21. In other words, in the graph V21, the number of people for each value of the maximum oxygen intake within a predetermined range is expressed by a bar graph.

In the variable display region V2, a mark V22 is set on the lower part of the graph V21. The mark V22 has a function of indicating a position corresponding to an estimated value of the maximum oxygen intake of the user. In other words, a position corresponding to an estimated value of the maximum oxygen intake of the user is indicated by the mark V22. In the variable display region V2, an estimated value (V23) of the maximum oxygen intake of the user is displayed on the lower part of the mark V22. Consequently, the user can recognize the estimated value of the maximum oxygen intake of their own, and the position of the estimated value of the maximum oxygen intake of their own in the group having the same age and sex as those of the user.

### [Change Result Screen]

FIG. 10 is a diagram illustrating an example of the change result screen ES3.

The change result screen ES3 is a screen included in the execution screens ES, and is a screen indicating a change in an estimated value of the maximum oxygen intake in a predetermined period (for example, for four months) and a change in a full marathon expected time. Specifically, the presentation information generation portion 370 generates change information regarding an estimated value of the maximum oxygen intake on the basis of an estimated value of the maximum oxygen intake on the date and time designated by the user or on a week including the date and time. The presentation information generation portion 370 generates change information regarding a full marathon expected time of the user calculated by the index calculation portion 369. In other words, the change information regarding an estimated value of the maximum oxygen intake and the change information of a full marathon expected time generated in the above-described way are displayed on the change result screen ES3 in correlation with the date and time (the date and time on which the maximum oxygen intake is estimated) on which the estimated value of the maximum oxygen intake is calculated. As illustrated in FIG. 10, in the change result screen ES3, fixed display regions F1 and F2 are respectively set on a screen upper part and a screen lower part, and a variable display region V3 is set therebetween.

A graph V31 representing the change information regarding an estimated value of the maximum oxygen intake of the user and the change information regarding a full marathon expected time is displayed in the variable display region V3. As illustrated in FIG. 10, a full marathon expected time is set on a left longitudinal axis of the group V31, and this indicates that the expected time is reduced toward the lower part of the graph V31. A value of the maximum oxygen intake is set on a right longitudinal axis of the graph V31, and this indicates that the value of the maximum oxygen intake increases toward the upper part of the graph V31. The date and time to which an estimated value of the maximum oxygen intake and a full marathon expected time of the user belong is set on a transverse axis of the graph V31.

Specifically, the graph V31 displays estimated values of the maximum oxygen intake and full marathon expected times of the user on Sep. 5, 2014, Oct. 5, 2014, Nov. 5, 2014, and Dec. 5, 2014. Specifically, an estimated value V311 of the maximum oxygen intake of the user on Sep. 5, 2014 is 30 ml/kg/min, and a full marathon expected time V315 is 4.5 hours. An estimated value V312 of the maximum oxygen intake of the user on Oct. 5, 2014 is 40 ml/kg/min, and a full marathon expected time V316 is 4.25 hours.

An estimated value V313 of the maximum oxygen intake of the user on Nov. 5, 2014 is 40 ml/kg/min, and a full marathon expected time V317 is 4.25 hours. Finally, an estimated value V314 of the maximum oxygen intake of the user on Dec. 5, 2014 is 50 ml/kg/min, and a full marathon expected time V318 is 3.9 hours.

An approximate straight line V319 for the full marathon expected times V315 to V318 is displayed in the variable display region V3. Consequently, the user can easily recognize changes in the estimated values V311 to V314 of the maximum oxygen intake of the user, changes in the full marathon expected times V315 to V318 of their own, and the approximate straight line V319 for four months.

### [Message Display Process in Detection Apparatus]

FIG. 11 is a diagram illustrating an example a message display screen SC1 displayed on the display section 241 of the detection apparatus 2.

If an estimated value of the maximum oxygen intake which has been estimated and message information including a message corresponding to the estimated value are received from the information processing apparatus 3, the detection apparatus 2 displays the message information on the display section 241 as illustrated in FIG. 11. Specifically, the message display screen SC1 is set on the display section 241, and the estimated value of the maximum oxygen intake of the user and the text that “there is concern that heart disease may occur; let's perform exercise!” are displayed on the message display screen SC1. The sound output section 242 of the detection apparatus 2 outputs a sound indicating that the message is displayed, and the vibration section 243 vibrates.

There may be a configuration in which a user is notified of such a message, for example, in a case where the user having no exercise habit, that is, the user whose exercise ability is not high suddenly continues to perform vigorous exercise. For example, this corresponds to a situation in which a user performs exercise (exercise of two ranks or three ranks higher) corresponding to an index value (exercise habit level) higher than a corresponding index value (exercise habit level) by a predetermined value or greater. In this case, for example, an estimated value of the maximum oxygen intake of the user and the text that “reduce the exercise intensity! you are in danger!” may be displayed on the message display screen SC1. Consequently, the user can be aware of performing exercise corresponding to exercise ability of their own or more, and can recognize that there is concern that the exercise may cause heart disease.

The message display screen SC1 may be displayed on the display unit 33 of the information processing apparatus 3.

### [Effects of First Embodiment]

As mentioned above, the biological information analysis system 1 according to the first embodiment achieves the following effects.

According to the biological information analysis system 1 of the present embodiment, it is possible to determine an exercise habit level of a user on the basis of biological information and body motion information of the user, and it is possible to calculate an estimated value of the maximum oxygen intake by employing a method of calculating an estimated value of the maximum oxygen intake using the above Jackson's formula on the basis of the determined exercise habit level and user information. Therefore, it is possible to easily calculate an estimated value of the maximum oxygen intake of a user without performing exercise applying a load to the user in a state of mounting a mask as in the gas phase respiratory function examination system disclosed in PTL 1. Since the estimated value can be calculated even without using a treadmill, it is possible to calculate the estimated value of the maximum oxygen intake of the user with a relative simple configuration.

In the gas phase respiratory function examination system disclosed in PTL 1, a user is required to perform exercise after wearing a mask in order to measure the maximum oxygen intake, and thus sanitary problems may occur. In contrast, in the present embodiment, it is not necessary to wear a mask, and thus it is possible to calculate an estimated value of the maximum oxygen intake sanitarily.

Since a distribution of values of the maximum oxygen intake in a group having the same age and sex as those of a user is presented, it is possible to easily recognize the distribution in the group. If distributions of values of the maximum oxygen intake in a plurality of groups are presented, a user can compare the distributions of values of the maximum oxygen intake in the respective groups with each other.

Since the change result screen ES3 including an estimated value of the maximum oxygen intake of a user is displayed on the display unit 33, the user has only to check the display unit 33, and can thus recognize the estimated value of the maximum oxygen intake of their own and a change in the estimated value of the maximum oxygen intake.

The user can recognize a position corresponding to the estimated value of the maximum oxygen intake of the user in a distribution of values of the maximum oxygen intake in a group having the same age and sex as those of the user. Consequently, the user can objectively recognize the standing position of their own, that is, the estimated value of the maximum oxygen intake of their own in the group. Therefore, it is possible to increase an exercise motivation of the user.

According to the present embodiment, since a full marathon expected time which is an exercise ability index of a user and change information regarding the expected time can be presented on the change result screen ES3, even a user not having the detailed knowledge about the maximum oxygen intake can easily recognize the exercise ability of their own. Consequently, it is possible to further increase an exercise motivation of the user.

According to the present embodiment, a height and a weight of a user which are input by operating the user information registration screen ES1 can be acquired, and thus a BMI can be calculated. Consequently, even if a value of the BMI cannot be acquired as user information, it is possible to reliably calculate an estimated value of the maximum oxygen intake by using the above Jackson's formula. Therefore, it is possible to improve a user's convenience.

According to the present embodiment, regarding the information processing apparatus 3, biological information and body motion information of a user are detected by the detection apparatus 2 and are then transmitted to the information processing apparatus 3, and thus the information processing apparatus 3 is not required to be provided with the detection unit 22 detecting the biological information and the body motion information. Therefore, since a user wears the detection apparatus 2, and can acquire biological information and body motion information for calculating an exercise habit level of the user, it is possible to more easily calculate an estimated value of the maximum oxygen intake than in a case where the biological information and the body motion information are acquired through an input operation performed by the user.

### Second Embodiment

Next, a description will be made of a second embodiment of the invention.

A biological information analysis system according to the present embodiment has the same configuration as that of the biological information analysis system 1, but is different from the biological information analysis system 1 in that the same detection apparatus and information processing apparatus as the detection apparatus 2 and the information processing apparatus 3 forming the biological information analysis system are provided in plurality, and the plurality of image processing apparatuses are connected to a server via a network. In the following description, the same portions or the substantially same portions as portions having already been described are given the same reference numerals, and description thereof will be omitted.

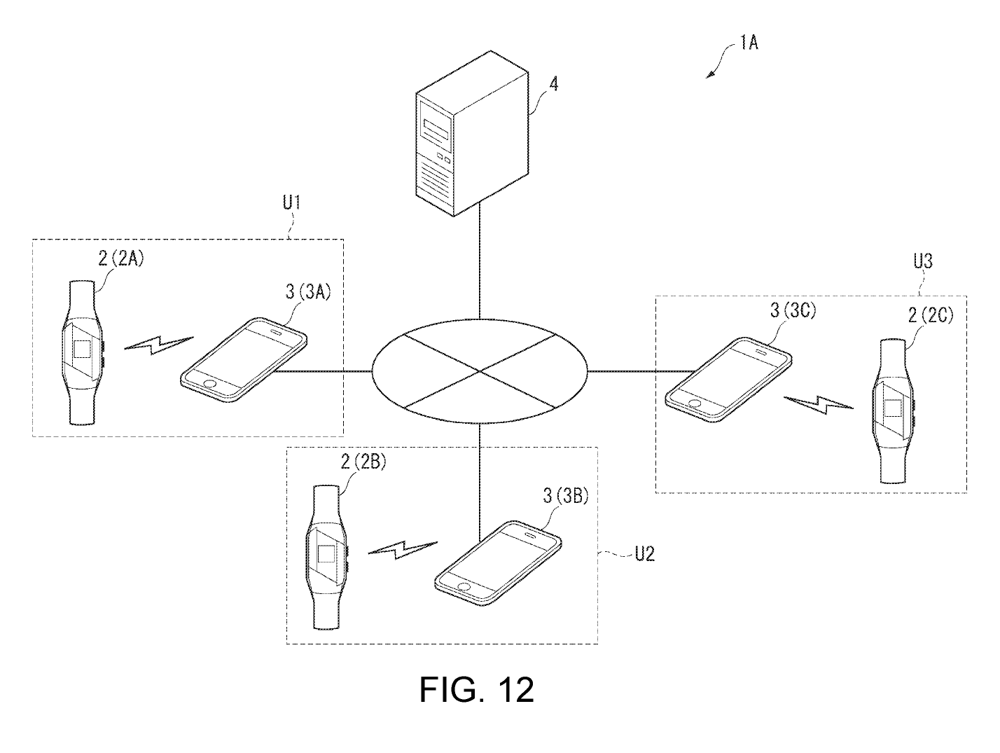

FIG. 12 is a schematic diagram illustrating a biological information analysis system 1A according to the present embodiment.

The biological information analysis system 1A according to the present embodiment includes a detection apparatus 2 (2A) and an information processing apparatus 3 (3A) used by a user U1, a detection apparatus 2 (2B) and an information processing apparatus 3 (3B) used by a user U2, a detection apparatus 2 (2C) and an information processing apparatus 3 (3C) used by a user U3, and a server 4.

Each of the plurality of detection apparatuses 2A to 2C has the same configuration as that of the detection apparatus 2. Each of the plurality of information processing apparatuses 3A to 3C has the same configuration as that of the information processing apparatus 3. The plurality of detection apparatuses 2A to 2C are respectively connected to the information processing apparatuses 3A to 3C through wireless communication in the same manner as in the detection apparatus 2 and the information processing apparatus 3. The plurality of information processing apparatuses 3A to 3C are connected to the server 4 via a network.

The server 4 is connected to the information processing apparatuses 3A to 3C, and has a function of acquiring not only user information of the users U1 to U3 but also estimated values of the maximum oxygen intake estimated (calculated) by the information processing apparatuses 3A to 3C, from the information processing apparatuses 3A to 3C. The server 4 generates a graph representing positions corresponding to estimated values of the maximum oxygen intake of the users U1 to U3 and all users (hereinafter, referred to as other users Un) using information processing apparatuses other than the information processing apparatuses 3A to 3C on the basis of user information and the estimated values of the maximum oxygen intake acquired from the respective information processing apparatuses 3A to 3C. In other words, in the present embodiment, the server 4 functions as the presentation information generation portion 370, and generates an analysis result screen ES4.

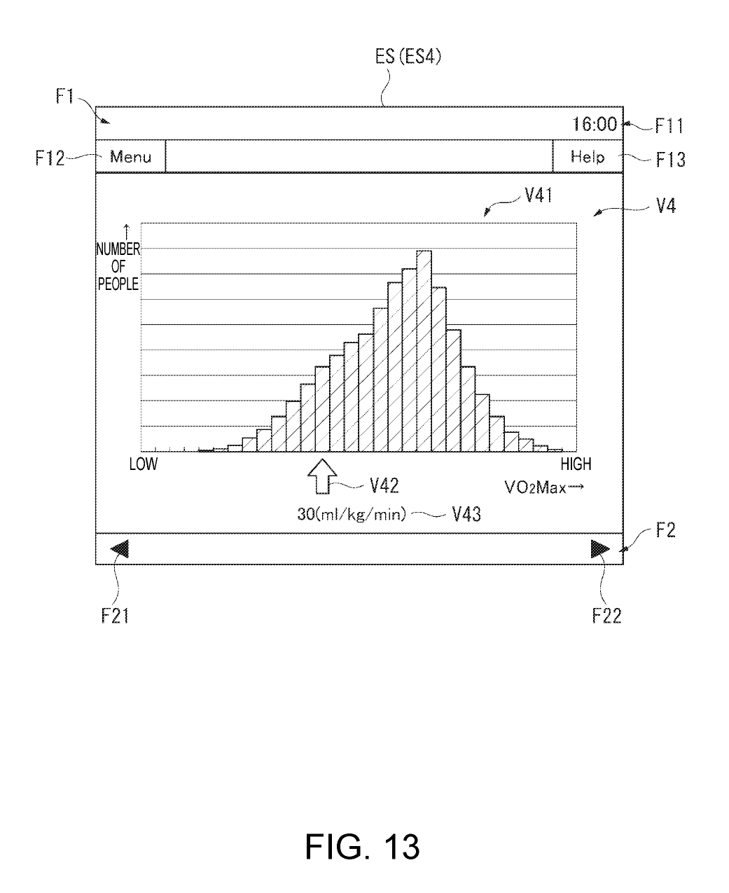

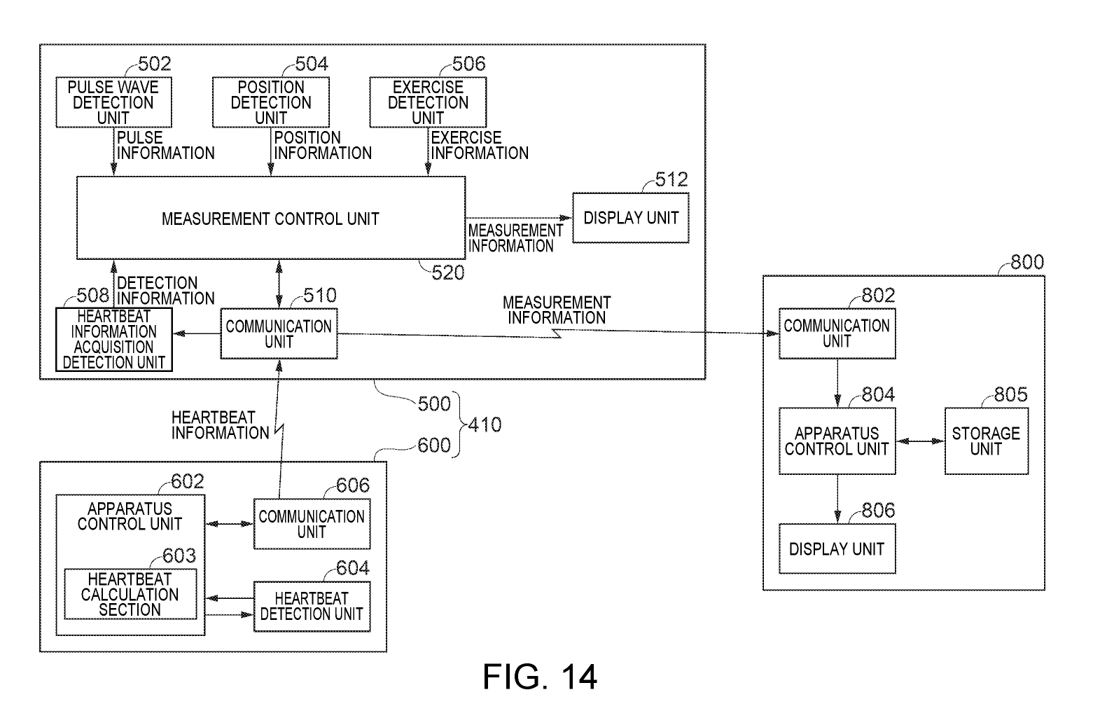

FIG. 13 is a diagram illustrating an example of the analysis result screen ES4. The analysis result screen ES4 includes estimated values of the maximum oxygen intake of the other users Un in addition to estimated values of the maximum oxygen intake of the respective users U1 to U3.

The analysis result screen ES4 is a screen included in the execution screens ES, and is a screen indicating a position corresponding to an estimated value of the maximum oxygen intake of a user (for example, the user U1) estimated by the estimated value calculation portion 368 in a distribution of estimated values of the maximum oxygen intake of the users U1 to U3 and the other users Un. In other words, in the present embodiment, the analysis result screen ES4 indicates a position corresponding to an estimated value of the maximum oxygen intake of a user in the distribution in the group of users using the same kind of apparatuses (the detection apparatuses 2 and the information processing apparatuses 3) in a period after starting of daily exercise. Specifically, the analysis result screen ES4 indicates a position corresponding to an estimated value of the maximum oxygen intake of the user in a distribution of estimated values of the maximum oxygen intake of the users U1 to U3 and the other users Un.

As illustrated in FIG. 13, in the analysis result screen ES4, fixed display regions F1 and F2 are respectively set on a screen upper part and a screen lower part, and a variable display region V2 is set therebetween.

A graph V41 representing a distribution of estimated values of the maximum oxygen intake of the users U1 to U3 and the other users Un is displayed in the variable display region V4. As illustrated in FIG. 13, the number of people of the group is set on a longitudinal axis of the graph V41, and this indicates that the number of people increases toward the upper part of the graph V41. An estimated value of the maximum oxygen intake is set on a transverse axis of the graph V41, and this indicates that the estimated value of the maximum oxygen intake increases toward the right of the graph V41. In other words, in the graph V41, the number of people for each estimated value of the maximum oxygen intake within a predetermined range is expressed by a bar graph.

In the variable display region V4, a mark V42 is set on the lower part of the graph V41. The mark V42 has a function of indicating a position corresponding to an estimated value of the maximum oxygen intake of a user.

In other words, a position corresponding to an estimated value of the maximum oxygen intake of a user is indicated by the mark V42. In the variable display region V4, an estimated value (V43) of the maximum oxygen intake of a user is displayed on the lower part of the mark V42. Consequently, a user (for example, the user U1) can recognize an estimated value (V43) of the maximum oxygen intake of their own, and a position of the estimated value of the maximum oxygen intake of their own in the users U1 to U3 and the other users Un.

### [Effects of Second Embodiment]

According to the present embodiment, the same effects as in the first embodiment are achieved, and the following effects are also achieved.

In the present embodiment, a position corresponding to an estimated value of the maximum oxygen intake of a user in a distribution of estimated values of the maximum oxygen intake of the users U1 to U3 and the other users Un is indicated by the mark V42 on the analysis result screen ES4. Consequently, the user can objectively recognize the position corresponding to the estimated value of the maximum oxygen intake of the user (user U1) in the users U2 and U3 and the other users Un. Therefore, it is possible to further increase an exercise motivation of the user.

### Third Embodiment

FIG. 14 is a block diagram illustrating a schematic configuration of a beating information measurement system 410. The beating information measurement system 410 has a function of measuring and displaying beating information indicating a pulse rate or a heart rate of a user as a subject, and is configured to include a pulse wave measurement apparatus 500 and a heartbeat measurement apparatus 600 which are communicably connected to each other. The pulse wave measurement apparatus 500 corresponds to a beating information measurement apparatus.

In the third embodiment, the pulse wave measurement apparatus 500 has an aspect of a wristwatch, and has a function of detecting a pulse signal corresponding to a pulse of a user if the user wears the pulse wave measurement apparatus on the wrist as a limb part. The heartbeat measurement apparatus 600 is a so-called heart rate monitor and is fixed to a chest belt, and has a function of outputting heartbeat information according to a heartbeat of the user if the user wears the chest belt on the chest which is a trunk part.

In measurement of a pulse, it is known that, in a case where a user performs vigorous exercise, an error easily occurs in pulse measurement in the arm due to noise, and, on the other hand, heartbeat measurement at a location close to the heart can be stably performed. The beating information measurement system 410 has a function of switching between measurement based on a pulse and measurement based on a heartbeat according to a user's exercise amount.

### [Configuration of Pulse Wave Measurement Apparatus]

First, the pulse wave measurement apparatus 500 will be described.

The pulse wave measurement apparatus 500 includes a pulse wave detection unit 502, a position detection unit 504, an exercise detection unit 506, a heartbeat information acquisition detection unit 508, a communication unit 510, a display unit 512, and a measurement control unit 520.

The pulse wave detection unit 502 acquires a pulse wave signal indicating a pulse wave of a user wearing the pulse wave measurement apparatus 500, generates pulse information (pulse wave information) based on the acquired pulse wave signal, and sends the generated pulse information to the measurement control unit 520.

The position detection unit 504 detects position information indicating a position where the pulse wave measurement apparatus 500 is located, and sends the detected position information to the measurement control unit 520.

The exercise detection unit 506 detects exercise information regarding motion (exercise) of the user wearing the pulse wave measurement apparatus 500, and sends the detected exercise information to the measurement control unit 520.

The communication unit 510 transmits and receives information to and from an external apparatus through wireless communication.

The heartbeat information acquisition detection unit 508 sends detection information to the measurement control unit 520 in a case where heartbeat information is acquired from the heartbeat measurement apparatus 600.

The measurement control unit 520 selects one of the pulse information sent from the pulse wave detection unit 502 and the heartbeat information sent from the heartbeat measurement apparatus 600 on the basis of the position information sent from the position detection unit 504 and the exercise information sent from the position detection unit 504, determines beating information on the basis of the selected one information, and sends the determined beating information to the display unit 512 so as to display the beating information.

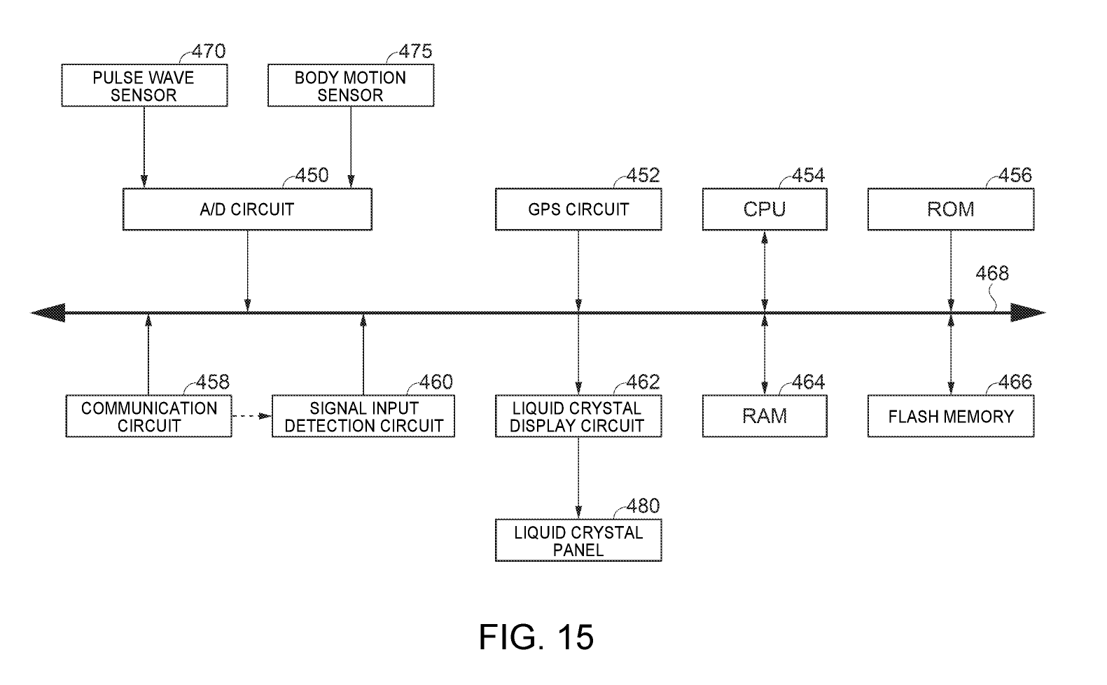

FIG. 15 is a diagram illustrating a hardware configuration of the pulse wave measurement apparatus 500. The pulse wave measurement apparatus 500 includes, as hardware, an A/D circuit 450, a GPS circuit 452, a CPU 454, a read only memory (ROM) 456, a communication circuit 458, a signal input detection circuit 460, a liquid crystal display circuit 462, a random access memory (RAM) 464, and a flash memory 466. This kind of hardware is connected to each other via bus 468. The A/D circuit 450 which converts an analog signal into a digital signal is connected to a pulse wave sensor 470 and a body motion sensor 475. The liquid crystal display circuit 462 is connected to a liquid crystal panel 480.

The pulse wave sensor 470 forms the pulse wave detection unit 502. The pulse wave sensor 470 includes a light source such as a light emitting diode (LED), and a light receiving element such as a phototransistor although not illustrated. The pulse wave sensor 470 applies light toward a skin surface of the user's wrist or the like from the light source, and receives reflected light in which the light is reflected at a blood vessel under the skin and is returned, with the light receiving element.

The reflected light from the blood vessel reflects a change in a blood flow due to a light absorption action of hemoglobin in blood, and thus intensity of their own changes. The pulse wave sensor 470 causes the light source to blink in a predetermined cycle shorter than a cycle of beating, and the light receiving element outputs a pulse wave signal (pulse information) corresponding to light reception intensity through photoelectric conversion whenever the light source is lit.

The body motion sensor 475 forms the exercise detection unit 506. The body motion sensor 475 includes at least acceleration sensors which output acceleration signals (exercise information) based on acceleration changes in multi-axis (for example, three axes) directions orthogonal to each other.

The A/D circuit 450 converts the pulse wave signal output from the pulse wave sensor 470 and the acceleration signals output from the body motion sensor 475 into digital signals.

The GPS circuit 452 forms the position detection unit 504. The GPS circuit 452 receives a radio wave signal from a geodetic satellite such as a GPS satellite rotating around the earth, and outputs a position signal (position information) regarding a location position on the basis of the received radio wave signal.

The communication circuit 458 forms the communication unit 510, and performs communication in a wireless manner according to a protocol of a short-range radio communication standard such as WiFi (registered trademark) or Bluetooth (registered trademark).

The signal input detection circuit 460 forms the heartbeat information acquisition detection unit 508, and detects that the communication circuit 458 receives heartbeat information transmitted from the heartbeat measurement apparatus 600.

The liquid crystal display circuit 462 and the liquid crystal panel 480 form the display unit 512. The liquid crystal display circuit 462 performs signal processes in order to display an input image signal as an image on the liquid crystal panel 480.

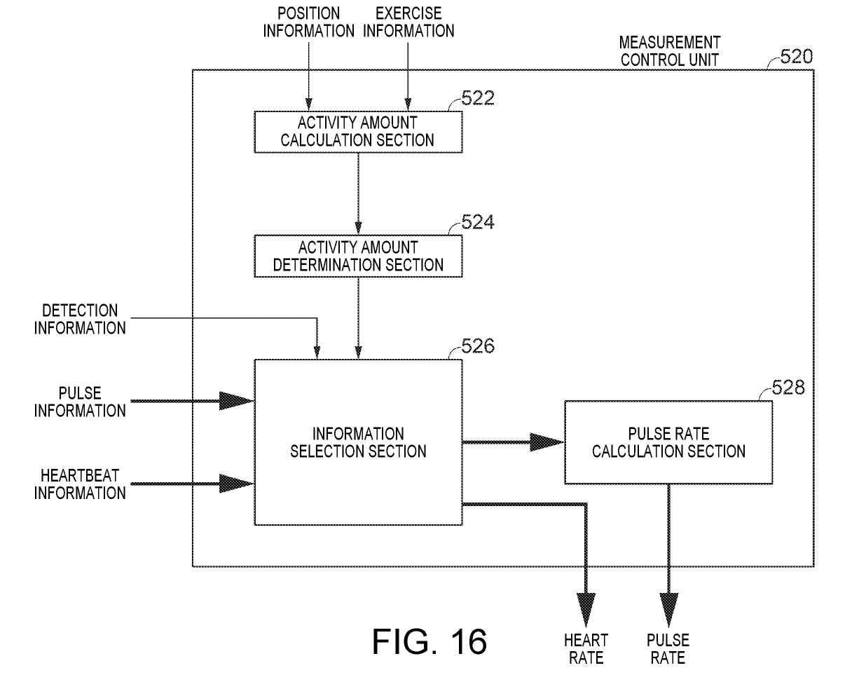

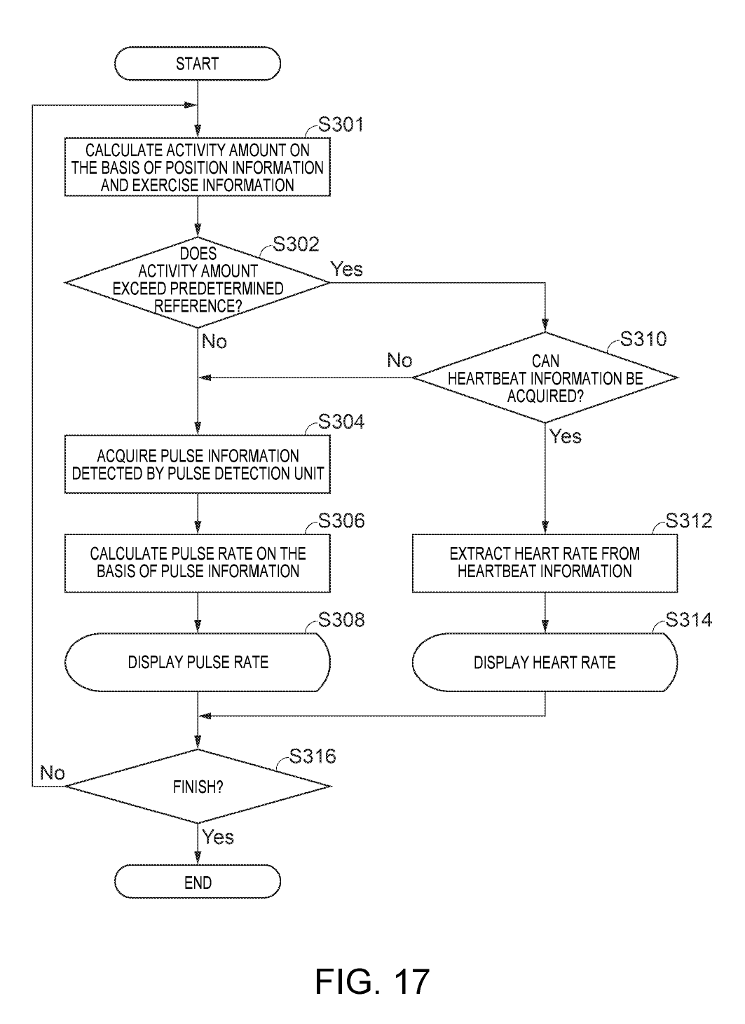

FIG. 16 is a diagram illustrating a detailed functional configuration of the measurement control unit 520. The measurement control unit 520 includes an activity amount calculation section 522, an activity amount determination section 524, an information selection section 526, and a pulse rate calculation section 528. The activity amount determination section 524 and the information selection section 526 correspond to a beating information determination unit.

The activity amount calculation section 522 calculates an activity amount regarding a user's body on the basis of the position information sent from the position detection unit 504 and the exercise information sent from the exercise detection unit 506.

In the third embodiment, examples of the calculated activity amount may include a movement amount, a movement speed, and exercise intensity of a user.

The activity amount calculation section 522 calculates a movement amount or a movement speed of a user by using a change in position information or the required time for movement. The activity amount calculation section 522 performs frequency analysis on acceleration signals as exercise information so as to calculate exercise intensity. As a method of calculating exercise intensity on the basis of acceleration signals, a method disclosed in, for example, JP-A-2012-232010 may be employed. The activity amount calculation section 522 calculates information regarding the movement amount, movement speed, and exercise intensity and the information is sent to the activity amount determination section 524.

The activity amount determination section 524 determines whether or not an activity amount of the user exceeds a predetermined reference value on the basis of the information sent from the activity amount calculation section 522.

In the third embodiment, it is determined whether or not at least one of a movement amount, a movement speed, and exercise intensity sent from the activity amount calculation section 522 exceeds a predetermined reference value, but this aspect is only an example. For example, it may be determined whether or not at least two of a movement amount, a movement speed, and exercise intensity exceed predetermined reference values, and a three-dimensional table including a movement amount, a movement speed, and exercise intensity may be created, and it may be determined whether or not the table is included in a reference three-dimensional region.

The predetermined reference value may be a fixed value which is set in advance, and may be a value which changes depending on the sex, the age, or the like of a user.

Information regarding the determination result in the activity amount determination section 524 is sent to the information selection section 526.

The information selection section 526 selects one of a pulse rate and a heart rate as beating information. In other words, the information selection section 526 selects one of calculating beating information on the basis of the pulse information output from the pulse wave detection unit 502 and using a heart rate detected by a heartbeat detection unit 604 as beating information.

In the third embodiment, in a case where the activity amount determination section 524 determines that the activity amount of the user does not exceed the predetermined reference value, the information selection section 526 selects calculation of beating information based on pulse information. In a case where the activity amount determination section 524 determines that the activity amount of the user exceeds the predetermined reference value, and the detection information sent from the heartbeat information acquisition detection unit 508 indicates acquisition of heartbeat information, the information selection section 526 selects a heart rate as beating information.

On the other hand, in a case where the activity amount determination section 524 determines that the activity amount of the user is higher than a predetermined level, and the detection information sent from the heartbeat information acquisition detection unit 508 indicates non-acquisition of heartbeat information, the information selection section 526 selects calculation of beating information based on pulse information.

In a case where calculation of beating information based on pulse information is selected, the information selection section 526 sends the pulse information to the pulse rate calculation section 528. On the other hand, in a case where a heart rate is selected as beating information, the information selection section 526 sends heartbeat information to the display unit 512 as measurement information so as to display the heartbeat information.

Generally, a pulse rate is the same as a heart rate as long as a pulse loss such as arrhythmia does not occur. Therefore, in the third embodiment, a heart rate calculated on the basis of a heartbeat signal may be treated as a pulse rate.

The pulse rate calculation section 528 calculates a pulse rate per reference time (for example, one minute) on the basis of an interval of pulse waves included in the pulse information.

The pulse rate calculation section 528 sends the calculated beating information to the display unit 512 as measurement information so as to display the beating information. Information indicating whether a pulse rate is calculated on the basis of a pulse wave signal or a heart rate is used may be included in measurement information so as to be displayed on the display unit 512.

The measurement control unit 520 may include a calorie amount calculation portion which calculates a calorie amount of a user in addition to beating information, and may display a calculated calorie amount as a calorie consumption amount of the user. A method of computing an oxygen intake on the basis of a pulse rate or a heart rate and converting the oxygen intake into a calculation consumption amount may employ a method disclosed in, for example, JP-A-2014-195711.

### [Configuration of Heartbeat Measurement Apparatus]

Referring to FIG. 14 again, a configuration of the heartbeat measurement apparatus 600 will be described.

The heartbeat measurement apparatus 600 includes an apparatus control unit 602, a communication unit 606, and the heartbeat detection unit 604.

The apparatus control unit 602 controls functions of the communication unit 606 and the heartbeat detection unit 604. The apparatus control unit 602 includes a heartbeat calculation section 603, calculates a heart rate on the basis of an interval of heartbeats, and sends heartbeat information including the calculated heart rate to the communication unit 606.

The heartbeat detection unit 604 detects a heartbeat on the basis of a change in an electrocardiographic signal which is input to electrodes in a state in which a plurality of electrodes are in contact with or electrostatically coupled to the chest.

A method in which a plurality of electrodes are in contact with or electrostatically coupled to the chest, and thus an electrocardiographic signal is acquired is disclosed in, for example, JP-A-2010-14169.

The communication unit 606 transmits heartbeat information to the communication unit 510 through wireless communication. In the third embodiment, in a case where the heartbeat detection unit 604 can calculate a heart rate, the apparatus control unit 602 starts to transmit heartbeat information.

### [Process in Information Terminal Apparatus]

In the third embodiment, there may be an aspect in which measurement information obtained through measurement in the heartbeat measurement apparatus 600 is transmitted to an information terminal apparatus 800.

The information terminal apparatus 800 may be a highly functional mobile phone such as a smart phone or a multi-function portable terminal such as a tablet PC, including a communication unit 802, an apparatus control unit 804, a storage unit 805, and a display unit 806.

Software for processing measurement information is incorporated into the information terminal apparatus 800. Therefore, the information terminal apparatus 800 stores measurement information transmitted from the heartbeat measurement apparatus 600 in the storage unit 805, reads the stored measurement information so as to perform a statistical process thereon as necessary, and displays a statistical result on the display unit 806. There may be an aspect in which the information terminal apparatus 800 further transmits the received measurement information or the like to an external server or the like via, for example, a public line.

### [Process in Pulse Wave Measurement Apparatus]

Next, FIG. 17 is a flowchart illustrating a flow of a beating information measurement process in the pulse wave measurement apparatus 500.

If the process is started, the CPU 454 calculates an activity amount on the basis of position information and exercise information of a user (step S301) <activity amount calculation step>.

Next, the CPU 454 determines whether or not the activity amount exceeds a predetermined reference (step S302) <selection step>.

Here, in a case where it is determined that the activity amount exceeds the predetermined reference (Yes in step S302), the CPU 454 determines whether or not heartbeat information can be acquired from the heartbeat measurement apparatus 600 (step S310).

Here, in a case where it is determined that the heartbeat information can be acquired from the heartbeat measurement apparatus 600 (Yes in step S310), the CPU 454 acquires the heartbeat information, and extracts a heart rate from the acquired heartbeat information (step S312) <heartbeat detection step>.

Next, the CPU 454 displays the heart rate as beating information, and proceeds to step S316.

In a case where it is determined that the activity amount does not exceed the predetermined reference (No in step S302), or it is determined that the heartbeat information cannot be acquired from the heartbeat measurement apparatus 600 (No in step S310), the CPU 454 acquires pulse information output from the pulse wave detection unit 502 (step S304) <pulse wave detection step>.

Next, the CPU 454 calculates a pulse rate on the basis of the acquired pulse information (step S306).

Next, the CPU 454 displays the calculated pulse rate as beating information (step S308), and proceeds to step S316.

In step S316, it is determined whether or not the CPU 454 receives a finish instruction (step S316), and the measurement process is finished in a case where it is determined that the finish instruction is received (Yes in step S316).

On the other hand, in a case where it is determined that the finish instruction is not received (No in step S316), the CPU returns to step S301 so as to repeatedly perform the process.

As a modification example of the measurement process in FIG. 17, in a case where the activity amount does not exceed the predetermined reference (No in step S302), the CPU 454 may be controlled to acquire both of the pulse information detected by the pulse wave detection unit 502 and the heartbeat information detected by the heartbeat measurement apparatus 600.

In this case, the pulse wave measurement apparatus 500 includes a first timing unit which generates a timing signal used as a reference of an operation timing for each constituent element of the pulse wave measurement apparatus 500, and performs a synchronization process for synchronization between the timing signal from the first timing unit with a second timing unit included in the heartbeat measurement apparatus 600, and thus the pulse wave measurement apparatus 500 is synchronized with the heartbeat measurement apparatus 600.

Beating information calculated on the basis of the pulse information and the heartbeat information may be displayed on the display unit 512. With this configuration, a user can understand a difference between a heartbeat and a pulse.

There may be a configuration in which the hardness of the artery of a user is estimated on the basis of pulse wave interval information calculated on the basis of a pulse wave signal detected by the pulse wave measurement apparatus 500 and a pulse wave interval information calculated on the basis of an electrocardiographic signal detected by the heartbeat measurement apparatus 600, and information indicating an estimation result is displayed on the display unit 512.

According to the above-described third embodiment, the following effects are achieved.

(1) The pulse wave measurement apparatus 500 mounted on a user's arm detects a pulse wave from the arm, and calculates a pulse rate on the basis of the detected pulse wave so as to display the pulse rate as beating information. The pulse wave measurement apparatus 500 calculates a user's activity amount, and, in a case of an activity situation in which the activity amount exceeds a predetermined reference, and in a case where the heartbeat measurement apparatus 600 is mounted on the chest of the user and thus a heartbeat is detected, the pulse wave measurement apparatus 500 displays a heart rate measured by the heartbeat measurement apparatus 600 as beating information. Therefore, the beating information measurement system 410 can measure accurate beating information even in a case where a user performs vigorous exercise.

(2) The activity amount calculation section 522 calculates an activity amount on the basis of not only a body motion but also position information, a movement amount and a movement speed which have been detected, and can thus calculate an accurate activity amount of a user.

### Fourth Embodiment

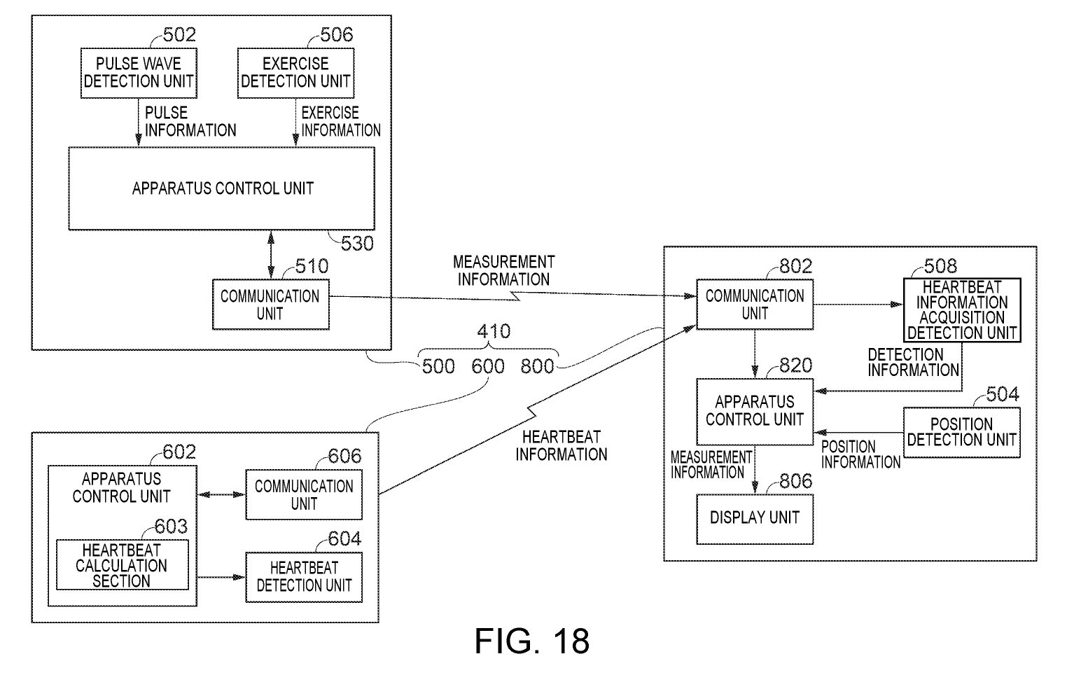

Next, with reference to FIG. 18, a fourth embodiment of the invention will be described.

In the third embodiment, the beating information measurement system 410 employs a configuration in which the pulse wave measurement apparatus 500 and the heartbeat measurement apparatus 600 are communicably connected to each other, but, in the fourth embodiment, the beating information measurement system 410 employs a configuration in which the pulse wave measurement apparatus 500, the heartbeat measurement apparatus 600, and the information terminal apparatus 800 are communicably connected to each other.

In other words, the heartbeat information acquisition detection unit 508, the position detection unit 504, and the measurement control unit 520 are provided in the pulse wave measurement apparatus 500 in the third embodiment, but are provided in the information terminal apparatus 800 in the fourth embodiment.

Therefore, the pulse wave measurement apparatus 500 transmits pulse information and exercise information to the information terminal apparatus 800 in response to an instruction from an apparatus control unit 530 which controls the functions.

In a case where a heart rate can be calculated, the heartbeat measurement apparatus 600 transmits heartbeat information to the information terminal apparatus 800.

An apparatus control unit 820 of the information terminal apparatus 800 acquires position information from the built-in position detection unit 504, selects one of pulse information and heartbeat information on the basis of the position information and exercise information, determines beating information on the basis of the selected information, and displays the determined beating information on the display unit 806.

There may be an aspect in which the apparatus control unit 820 holds the determined beating information, performs a statistical process for each user, and a process result is displayed on the display unit 806.

According to the above-described fourth embodiment, the same effects as in the third embodiment are achieved.

### [Modifications of Embodiments]

The invention is not limited to the embodiments, and modifications, alterations, and the like are included in the invention within the scope of being capable of achieving the object of the invention.

In the first embodiment, the change result screen ES3 is displayed on the display unit 33, and thus an estimated value of the maximum oxygen intake of a user and change information regarding the estimated value are displayed. However, the invention is not limited thereto. For example, change information regarding an estimated value of the maximum oxygen intake of a user may not be displayed on the display unit 33. The estimated value of the maximum oxygen intake may be transmitted to the detection apparatuses 2 and 2A to 2C, and may be displayed on the display sections 241 of the detection apparatuses 2 and 2A to 2C.

In the respective embodiments, the analysis result screen ES2 or ES4 is displayed on the display unit 33, and thus a position corresponding to a value of the maximum oxygen intake of a person satisfying a predetermined condition or an estimated value of the maximum oxygen intake of the user in a distribution of estimated values of the maximum oxygen intake is presented.

However, the invention is not limited thereto. For example, the analysis result screens ES2 and ES4 may not be displayed on the display unit 33. Only an estimated value of the maximum oxygen intake of a user may be displayed on the display unit 33 instead of the analysis result screens ES2 and ES4.

In the first embodiment, the analysis result screen ES2 is displayed on the display unit 33, and thus a position corresponding to an estimated value of the maximum oxygen intake of a user in a distribution of values of the maximum oxygen intake of a group having the same age and sex as those of the user is presented. However, the invention is not limited thereto. For example, a position corresponding to an estimated value of the maximum oxygen intake of a user in a distribution of values of the maximum oxygen intake of a group having the same sex as that of the user may be presented, and a position corresponding to an estimated value of the maximum oxygen intake of a user in a distribution of values of the maximum oxygen intake of a group having the same age as that of the user may be presented.

In the above-described respective embodiments, the graphs V21 and V41 and the marks V22 and V42 are presented on the analysis result screens ES2 and ES4, and thus a position corresponding to an estimated value of the maximum oxygen intake of a user in a distribution of values of the maximum oxygen intake of various groups is presented. However, the invention is not limited thereto. For example, the graphs V21 and V41 and the marks V22 and V42 may not be displayed. In this case, ranking (for example, 18-th ranking) of an estimated value of the maximum oxygen intake of a user in various groups may be displayed. Consequently, a user can more objectively recognize a position corresponding to an estimated value of the maximum oxygen intake of the user in a group.

In the first embodiment, the change result screen ES3 is displayed on the display unit 33, and thus the full marathon expected times V315 to V318 and the approximate straight line V319 are presented as exercise ability indexes of a user. However, the invention is not limited thereto. For example, the full marathon expected times V315 to V318 may not be presented. On the change result screen ES3, only the approximate straight line V319 may be displayed without displaying the full marathon expected times V315 to V318 for the respective dates. The full marathon expected times V315 to V318 for the respective dates may be displayed, and the approximate straight line V319 may not be displayed.

In the above-described respective embodiments, the index calculation portion 369 calculates a full marathon expected time as an exercise ability index indicating the exercise ability of a user on the basis of biological information, body motion information, an exercise habit level, user information, and an estimated value of the maximum oxygen intake. However, the invention is not limited thereto. For example, the index calculation portion 369 may calculate a full marathon expected time on the basis of only user information and an estimated value of the maximum oxygen intake of a user. A full marathon expected time may be calculated on the basis of at least any of biological information, body motion information, and an exercise habit level.

An exercise ability index of a user is not limited to the full marathon expected time. For example, the index calculation portion 369 may calculate an expected time for a half marathon or an expected time for 3,000 m running as an exercise ability index.

An exercise ability index is not limited to a time for the long distance running, and other indexes (for example, a level or a rank indicating exercise ability) may be used.

In the above-described respective embodiments, when a height and a weight are input on the user information registration screen ES1, a BMI is automatically calculated and registered. However, the invention is not limited thereto. For example, a user may input a BMI by himself/herself. In a case where a BMI is input earlier than a height and a weight, the height and the weight of a user may not be input.

In the above-described respective embodiments, the level determination portion 367 determines an exercise habit level on the basis of biological information and body motion information of a user for a week which is a predetermined period. However, the invention is not limited thereto. For example, the predetermined period may be two weeks, and may be four weeks. To summarize, the predetermined period may be any period as long as the period is one or more weeks.

In the above-described respective embodiments, an exercise habit level is determined on the basis of an exercise time of each zone. However, the invention is not limited thereto. For example, the analysis portion 366 may detect a running distance and a walking distance of a user, and the level determination portion 367 may determine an exercise habit level on the basis of the running distance and the walking distance.

In the above-described respective embodiments, the estimated value calculation portion 368 calculates an estimated value of the maximum oxygen intake by assigning an exercise habit level, and the age, the sex, and the BMI of a user to the so-called Jackson's formula. However, the invention is not limited thereto. For example, the estimated value calculation portion 368 is not limited to the Jackson's formula, and may calculate the maximum oxygen intake on the basis of the maximum heart rate and a heart rate in a stable state.

In the above-described respective embodiments, a pulse wave is detected as biological information. However, the invention is not limited thereto. For example, as biological information, a heart rate, a blood sugar level, and the like of a user may be detected in addition to the pulse wave, or may be detected instead of the pulse wave.

In the above-described respective embodiments, the detection apparatuses 2 and 2A to 2C are mounted on a user's wrist. However, the invention is not limited thereto. For example, the detection apparatuses 2 and 2A to 2C may be mounted on the ankle, the chest, or the like of a user.

In the above-described respective embodiments, the detection apparatus 2 includes the biological information detection section 221 detecting biological information. However, the invention is not limited thereto, and the biological information detection section 221 may not be provided. In this case, the body motion information detection section 222 may detect the number of steps as body motion information. With this configuration, the level determination portion 367 can determine an exercise habit level of a user on the basis of running and walking distances of the user.

The detection apparatus 2 may include an activity amount meter, calculate calorie consumption, and calculate an exercise habit level on the basis of the calorie consumption.

In the second embodiment, a position corresponding to an estimated value of the maximum oxygen intake of a user in a group including all users using the detection apparatuses 2A to 2C is displayed on the analysis result screen ES4. However, the invention is not limited thereto. For example, in a case where information sharing is set among the users U1 to U3 of the group, a position of each of the users U1 to U3 sharing information in the group, ranking of each user in all users, ranking of each user among the users U1 to U3, or the like may be displayed on the analysis result screen.

Each functional unit of the pulse wave measurement apparatus 500 illustrated in FIGS. 14, 16 and 18 indicates a functional configuration realized in cooperation between hardware and software, and a specific mounting aspect is not particularly limited. Therefore, there may be a configuration in which individually corresponding hardware is not necessarily mounted in each functional unit, and a single processor executes a program so as to realize functions of a plurality of functional units. In the above-described embodiments, some functions realized by software may be realized by hardware, or some functions realized by hardware may be realized by software. Specific detailed configurations of other respective units of the beating information measurement system 410 may also be arbitrarily changed within the scope without departing from the spirit of the invention.

### REFERENCE SIGNS LIST

1 AND 1A BIOLOGICAL INFORMATION ANALYSIS SYSTEM 2 AND 2A TO 2C BIOLOGICAL INFORMATION DETECTION APPARATUS (DETECTION APPARATUS), 3 AND 3A TO 3C INFORMATION PROCESSING APPARATUS (BIOLOGICAL INFORMATION ANALYSIS APPARATUS), 4 SERVER, 23 RECEPTION UNIT, 25 COMMUNICATION UNIT, 26 STORAGE UNIT, 27 CONTROL UNIT, 32 COMMUNICATION UNIT, 33 DISPLAY UNIT (RANGE PRESENTATION UNIT AND INDEX PRESENTATION UNIT), 35 STORAGE UNIT, 36 CONTROL UNIT, 221 BIOLOGICAL INFORMATION DETECTION SECTION, 222 BODY MOTION INFORMATION DETECTION SECTION, 275 INFORMATION ACQUISITION SECTION, 365 INFORMATION ACQUISITION PORTION, 366 ANALYSIS PORTION, 367 LEVEL DETERMINATION PORTION, 368 ESTIMATED VALUE CALCULATION PORTION (ESTIMATION UNIT), 369 INDEX CALCULATION PORTION, 370 PRESENTATION INFORMATION GENERATION PORTION, 410 BEATING INFORMATION MEASUREMENT SYSTEM, 450 A/D CIRCUIT, 452 GPS CIRCUIT, 454 CPU, 456 ROM, 458 COMMUNICATION CIRCUIT, 460 SIGNAL INPUT DETECTION CIRCUIT, 462 LIQUID CRYSTAL DISPLAY CIRCUIT, 464 RAM, 466 FLASH MEMORY, 468 BUS, 470 PULSE WAVE SENSOR, 475 BODY MOTION SENSOR, 480 LIQUID CRYSTAL PANEL, 500 PULSE WAVE MEASUREMENT APPARATUS, 502 PULSE WAVE DETECTION UNIT, 504 POSITION DETECTION UNIT, 506 EXERCISE DETECTION UNIT, 508 HEARTBEAT INFORMATION ACQUISITION DETECTION UNIT, 510 COMMUNICATION UNIT, 512 DISPLAY UNIT, 520 MEASUREMENT CONTROL UNIT, 522 ACTIVITY AMOUNT CALCULATION SECTION, 524 ACTIVITY AMOUNT DETERMINATION SECTION, 526 INFORMATION SELECTION SECTION, 528 PULSE RATE CALCULATION SECTION, 530 APPARATUS CONTROL UNIT, 600 HEARTBEAT MEASUREMENT APPARATUS, 602 APPARATUS CONTROL UNIT, 603 HEARTBEAT CALCULATION SECTION, 604 HEARTBEAT DETECTION UNIT, 606 COMMUNICATION UNIT, 800 INFORMATION TERMINAL APPARATUS, 802 COMMUNICATION UNIT, 804 APPARATUS CONTROL UNIT, 805 STORAGE UNIT, 806 DISPLAY UNIT, 820 APPARATUS CONTROL UNIT, ES EXECUTION SCREEN, ES1 USER INFORMATION REGISTRATION SCREEN, ES2 AND ES4 ANALYSIS RESULT SCREEN, ES3 CHANGE RESULT SCREEN

1. A biological information analysis apparatus comprising:
an information acquisition unit that acquires biological information of a user, body motion information indicating body motion of the user, and user information regarding the user;
a storage unit that stores the biological information, the body motion information, and the user information which have been acquired;
a level determination unit that determines an exercise habit level of the user on the basis of the biological information and the body motion information in a predetermined period; and
an estimation unit that estimates the maximum oxygen intake of the user on the basis of the exercise habit level and the user information.

2. The biological information analysis apparatus according to claim 1, further comprising:
a presentation unit that presents a distribution of values of the maximum oxygen intake in a group which is classified on the basis of the user information.

3. The biological information analysis apparatus according to claim 2,
wherein the presentation unit presents a position corresponding to an estimated value of the maximum oxygen intake of the user in the distribution.

4. The biological information analysis apparatus according to claim 1, further comprising:
an index calculation unit that calculates an exercise ability index indicating exercise ability of the user on the basis of the user information and the estimated value of the maximum oxygen intake; and
an index presentation unit that presents the calculated exercise ability index.

5. The biological information analysis apparatus according to claim 1, further comprising:
an index calculation unit that calculates an exercise ability index indicating exercise ability of the user on the basis of at least one of the biological information, the body motion information, and the exercise habit level; and
an index presentation unit that presents the calculated exercise ability index.

6. The biological information analysis apparatus according to claim 1 further comprising:
a change information presentation unit that presents a change in the estimated value of the maximum oxygen intake of the user in correlation with the date and time on which the maximum oxygen intake is estimated.

7. The biological information analysis apparatus according to claim 4, further comprising:
a change information presentation unit that presents a change in the exercise ability index along with a change in the estimated value of the maximum oxygen intake of the user in correlation with the date and time on which the maximum oxygen intake is estimated.

8. The biological information analysis apparatus according to claim 1,
wherein the user information includes the age, the sex, a height, and a weight of the user.

9. A biological information analysis system comprising:
the biological information analysis apparatus according to claim 1; and
a detection apparatus that is mounted on the user,
wherein the detection apparatus includes
a biological information detection unit that detects biological information of the user,
a body motion information detection unit that detects body motion information of the user, and
a detection apparatus side transmission unit that transmits the biological information and the body motion information to the biological information analysis apparatus.

10. A biological information analysis program which is read to be executed by a computer, the program causing the computer to function as:
the biological information analysis apparatus according to claim 1.

11. A beating information measurement system comprising:
a pulse wave detection unit that detects pulse wave information indicating a pulse wave;
a heartbeat detection unit that detects heartbeat information indicating a heartbeat;
an activity amount calculation unit that calculates an activity amount of the body; and
a beating information determination unit that selects one of the pulse wave information and the heartbeat information on the basis of the activity amount, and determines beating information on the basis of the selected information,
wherein the beating information determination unit determines the beating information on the basis of the heartbeat information in a case where the activity amount exceeds a predetermined reference.

12. The beating information measurement system according to claim 11,
wherein the beating information determination unit determines the beating information on the basis of the pulse wave information in a case where the activity amount does not exceed the predetermined reference.

13. The beating information measurement system according to claim 11,
wherein the beating information is a heart rate or a pulse rate.

14. The beating information measurement system according to claim 11,
wherein the beating information determination unit includes a determination section that determines whether or not the heartbeat detection unit detects the heartbeat information.

15. The beating information measurement system according to claim 14,
wherein the beating information determination unit determines the beating information on the basis of the heartbeat information in a case where the determination section determines that the heartbeat detection unit detects the heartbeat information, and the activity amount exceeds the predetermined reference.

16. The beating information measurement system according to claim 11,
wherein the activity amount calculation unit calculates the activity amount on the basis of position information based on a location position and exercise information indicating exercise intensity based on body motion.

17. The beating information measurement system according to claim 11, further comprising:
a display unit that displays the beating information,
wherein the beating information determination unit displays information indicating whether the beating information is calculated on the basis of the heartbeat information or is calculated on the basis of the pulse wave information, on the display unit.

18. The beating information measurement system according to claim 11, further comprising:
a calorie amount calculation unit that calculates a calorie consumption amount on the basis of the calculated beating information.
